(19) United States
(12) Patent Application Publication
(10) Pub. No.: US 2024/0277614 A1
(43) Pub. Date: Aug. 22, 2024

(54) LONG-ACTING FORMULATIONS

(71) Applicant: Janssen Pharmaceutica NV, Beerse (BE)

(72) Inventors: René HOLM, Titusville, NJ (US); Iwan Caroline F. VERVOORT, Hoogstraten (BE); Wenyu DONG, Den Haag (NL); Miriam COLOMBO, Antwerp (BE)

(21) Appl. No.: 18/004,464

(22) PCT Filed: Jul. 8, 2021
(86) PCT No.: PCT/EP2021/068957

§ 371 Date: Jan. 6, 2023

(30) Foreign Application Priority Data
Jul. 9, 2020 (EP) 20185106.0

(51) Int. Cl. A61K 9/10; A61K 9/00; A61K 31/4704; A61K 47/10; A61K 47/22

(52) U.S. Cl. CPC A61K 9/10; A61K 9/0019; A61K 31/4704; A61K 47/10; A61K 47/22

(57) ABSTRACT

This invention concerns pharmaceutical compositions for administration via intramuscular or subcutaneous injection, comprising micro- or nano-particles of an active ingredient, suspended in an aqueous pharmaceutically acceptable carrier, and comprising PEG4000 as a surface modifier, and the use of such pharmaceutical compositions in the treatment and prophylaxis of a pathogenic mycobacterial infection.

### FIELD OF THE INVENTION

This invention concerns pharmaceutical compositions for administration via intramuscular or subcutaneous injection, comprising micro- or nano-particles of an active pharmaceutical ingredient (API), suspended in an aqueous pharmaceutically acceptable carrier, and which contain PEG4000. It further relates to the sterilization of such compositions and subsequent re-suspension.

### BACKGROUND OF THE INVENTION

The goal of long-acting formulations can be to reduce drug burden. This is particularly useful for treatment regimens that may last several months.

The number and/or volume of dosage forms that need to be administered are commonly referred to as “pill burden”. A high pill burden is undesirable for many reasons, such as the frequency of intake, often combined with the inconvenience of having to swallow large dosage forms, as well as the need to store and transport a large number or volume of pills. A high pill burden increases the risk of patients not taking their entire dose, thereby failing to comply with the prescribed dosage regimen. As well as reducing the effectiveness of the treatment, this may also lead to the emergence of resistance (e.g. in the case of bedaquiline, bacterial resistance).

It would be attractive to provide therapy involving the administration of dosage forms at long time intervals such as one week or longer, or even one month or longer.

Various formulations are known in the art, including long-acting ones. For instance, micro- and nano-suspension technology is known for achieving long-acting formulations in the field of anti-HIV drugs, for instance as described in international patent applications WO 2007/147882 and WO 2012/140220. Further, nano-particles known in the prior art have been described, for example, in EP-A-0 499 299. Such particles have an average particle size in the submicron range and consist of particles of a crystalline drug substance having a surface modifier adsorbed on their surface. Nanoparticles have also been used to formulate poorly water-soluble active ingredients.

Long-acting formulations of the anti-tuberculosis drug bedaquiline are also described in international patent application WO 2019/012100.

A challenge relating to the manufacture and suitability of such long-acting formulations relates to fact that they have to be sterilized (which is important for injectables, for instance if they are intended to be administered intraveneously, intramuscularly or subcutaneously). There are a number of different ways to sterilize such long-acting formulations, including by heat sterilization, autoclaving and gamma-radiation (γ-radiation). An example of some methods are described in e.g. US patents/applications U.S. Pat. Nos. 5,298,262, 5,346,702 and US 2010/255102. For heat sterilization and autoclaving, it is important to be able to select excipients (e.g. surface modifiers or surfactants) that are autoclavable, e.g. do not degrade. Further challenges arise after such sterilization, which are linked to desired stability of the long-acting formulation, undesired aggregation of particles of the active pharmaceutical ingredient (API) within that formulation and the desired re-suspendability of the formulation (after sterilization, e.g. autoclaving). U.S. Pat. Nos. 5,298,262 and 5,346,702 disclose the use of cloud point modifiers to prevent particle aggregation during sterilization. The cloud point is the temperature above which the surfactant (or surface modifier) phase-separates and precipitates out of the solution. Heat sterilization or autoclaving of suspensions must be performed below the cloud point of the surfactant/surface modifier as otherwise they would phase-separate and precipitate when heated above their cloud temperature due to a solubility change. This would leave the particle (of the active pharmaceutical ingredient) surface free and the particles would thereby aggregate. The idea of a cloud point modifier (or booster) is to allow the temperature of the sterilization or autoclaving process to be higher and thereby preventing or limiting particle aggregation. The cloud point modifiers mentioned in U.S. Pat. Nos. 5,298,262 and 5,346,702 include ionic and nonionic cloud point modifiers, such as sodium dodecyl sulfate, dodecyltrimethyl-ammonium bromide, polyethylene glycol and propylene glycol. The polyethylene glycols mentioned as cloud point modifiers include PEG300, PEG400, PEG1000 and PEG2000, with PEG400 indicated as being preferred, and in the examples specifically PEG400 and PEG1000 were shown to raise cloud point (of Tetronic 908). Other cloud point modifiers or boosters are also described in a number of other documents.

Now further alternative and/or improved long acting formulations are described, and the invention relates to such formulations.

### SUMMARY OF THE INVENTION

The present invention is concerned with a pharmaceutical composition for administration by intramuscular or subcutaneous injection, comprising a therapeutically effective amount of an active pharmaceutical ingredient, or a pharmaceutically acceptable salt thereof, in the form of a suspension of micro- or nano-particles comprising:
(a) an active pharmaceutical ingredient, or a pharmaceutically acceptable salt thereof, in micro- or nanoparticle form, and a surface modifier; and
(b) a pharmaceutically acceptable aqueous carrier, which is characterised in that the surface modifier comprises PEG4000 or the like, and in which the composition undergoes sterilization (e.g. by autoclaving) and re-suspension,
   wherein such a composition may be referred to herein as “composition(s) of the invention”.

PEG4000, or, polyethylene glycol 4000, is a known high-molecular weight polymer where the 4000 refers to the approximate average molecular weight in daltons. PEG4000 is commercially available from sources such as Sigma-Aldrich and hence why it is used as such. However, embraced within the scope of the invention (e.g. when the term “PEG4000” or “PEG4000, or the like” is used) are other high-molecular weight polyethylene glycols, for instance those above 1000 and up to 8000 (e.g. PEG1000 to PEG8000, for instance PEG2000 to PEG6000), even though in a particular embodiment the PEG group when referred to herein in the context of the invention is PEG3000 to PEG5000 (e.g. PEG3500 to PEG4500). As indicated herein, the number next to the PEG represents average molecule weight in daltons, as it is understood that most PEGs include molecules with a distribution of molecular weights, i.e. they are polydisperse.

The composition of the invention is a suspension, by which we mean that the active pharmaceutical ingredient (or API) is suspended in the pharmaceutically acceptable aqueous carrier.

The composition of the invention (i.e. the suspension) contains a surface modifier, which may be adsorbed onto the surface of the active ingredient (or API). As indicated, the surface modifier comprises PEG4000, or the like (and may also contain other surface modifiers, such as those described hereinafter).

Active pharmaceutical ingredient refers to those which exert a pharmacological, immunological or metabolic action with a view to restoring, correcting or modifying physiological functions or to make a medical diagnosis. The active pharmaceutical ingredient may be any known active ingredient that is amenable to form micro- or nano-particles in the context of the compositions of the invention (suspensions) described herein. In an embodiment, such active pharmaceutical ingredient may be sterilized (e.g. by irradiation, heat sterilization or autoclaving) and, in a further embodiment such active pharmaceutical ingredient is autoclavable (for instance, the suspension containing such active pharmaceutical ingredient is autoclavable). In yet a further embodiment, the active pharmaceutical ingredient is re-suspending after sterilization (e.g. after autoclaving). A particular API that may be mentioned includes an antibiotic, antibacterial (e.g. antituberculosis) or antiviral drug (either approved or being developed), which meets the foregoing criteria (e.g. it may be micronized to form micro- or nano-particles, and a suspension containing it is autoclavable). A specific API that may be mentioned is the anti-tuberculosis drug bedaquiline, which has received marketing approval in a number of countries and is marketed under the trade name Sirturo® (which is bedaquiline, formulated as the fumarate salt).

In an embodiment, the present invention may therefore concern a pharmaceutical composition for administration by intramuscular or subcutaneous injection, comprising a therapeutically effective amount of an active pharmaceutical ingredient, or a pharmaceutically acceptable salt thereof, in the form of a suspension of micro- or nano-particles comprising:
(a) an active pharmaceutical ingredient, or a pharmaceutically acceptable salt thereof, in micro- or nanoparticle form, having a surface modifier adsorbed to the surface thereof, and
(b) a pharmaceutically acceptable aqueous carrier; wherein the active ingredient is suspended,
   and wherein the surface modifier comprises PEG4000, or the like, and in which the composition undergoes sterilization (e.g. by autoclaving) and re-suspension.

It will be understood that the term “long term treatment” refers to treatment where one dose or one administration (e.g. by intramuscular or subcutaneous injection) will have a persistent therapeutic effect over a time period, as described herein, for instance a persistent therapeutic effect over several hours, weeks or months (e.g. in an embodiment, over a period of at least or up to one month, three months or six months); see examples. Put another way, long term treatment may refer to, where there is more than one dose/administration, the long period of time (as described herein) between the doses/administrations, i.e. the intervals are a long period of time as described herein.

In another aspect, there is provided a method for the long term treatment of a subject with a particular disease or condition (e.g. that requires an antibiotic, antibacterial or antiviral drug), as described herein (e.g. above) wherein one dose or administration (e.g. of the amount described herein, e.g. hereinafter) is provided/required (and has a persistent effect, e.g. over a time period described herein). In another aspect, there is provided such a long term treatment regime, where two such doses or administrations are provided/required, which doses/administrations are given at intervals, wherein the interval time period is that as described herein, e.g. a period of at least or up to one month, three months or six months—for instance for a period of time in which persistent therapeutic effect lasts). In a further embodiment, there is provided such a long term treatment regime, in which three such doses or administrations are provided/required at such intervals as herein described. In yet a further embodiment, there is provided a long term treatment regime as herein described but which is preceded with a lead-in treatment phase (that is not a long term treatment regime, e.g. a once-daily administration course, lasting for one week, two weeks, three weeks or one month).

In another aspect the invention relates to a method for the long term prevention of a particular disease or condition in a subject at risk of such disease or condition, said method comprising administering to said subject an effective amount of a pharmaceutical composition as specified above or as further specified hereinafter, wherein the composition is administered or is to be administered intermittently at a time interval that is in the range of one week to one year, or one week to two years.

The present invention furthermore relates to the use of a pharmaceutical composition as specified above or as further specified hereinafter, for the manufacture of a medicament for the long term prevention for the long term prevention of a disease or condition in a subject at risk of such disease or condition, wherein the composition is administered or is to be administered intermittently at a time interval that is in the range of one week to one year or one week to two years.

In one embodiment the invention concerns a use or a method as specified herein, wherein the pharmaceutical composition is administered or is to be administered at a time interval that is in the range of one week to one month, or in the range of one month to three months, or in the range of three months to six months, or in the range of six months to twelve months, or in the range of 12 months to 24 months.

In another embodiment the invention concerns a use or a method as specified herein, wherein the pharmaceutical composition is administered or is to be administered once every two weeks, or once every month, or once every three months.

Further pharmaceutical compositions, methods of treatment or prevention, as well as uses for the manufacture of medicaments based on these compositions will be described hereinafter and are meant to be part of the present invention.

The invention is also described with reference to the following figures:

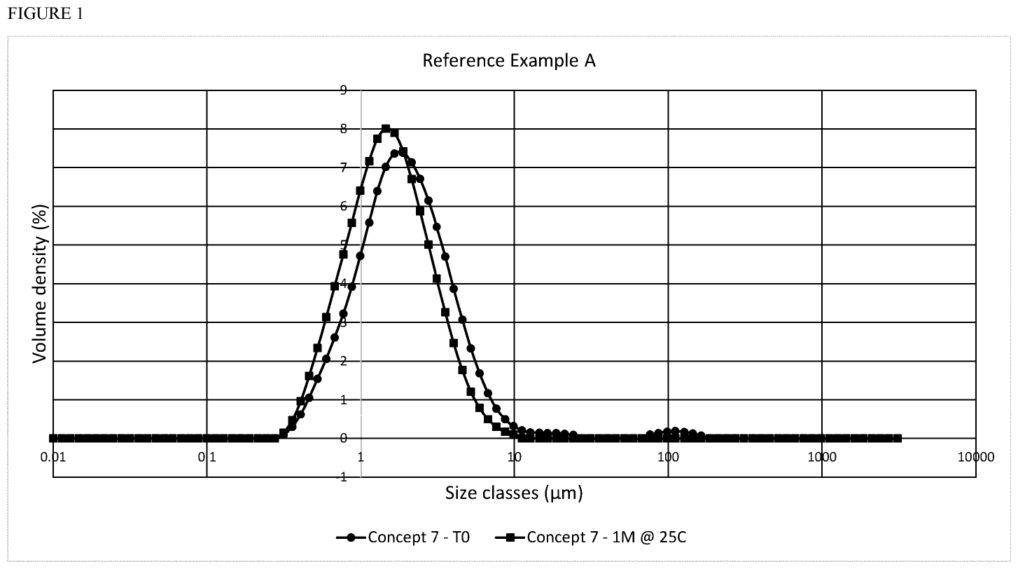

FIG. 1: PSD measurements of Reference Example A, at time zero and at 1 month, where “Concept 7” refers to Reference Example A

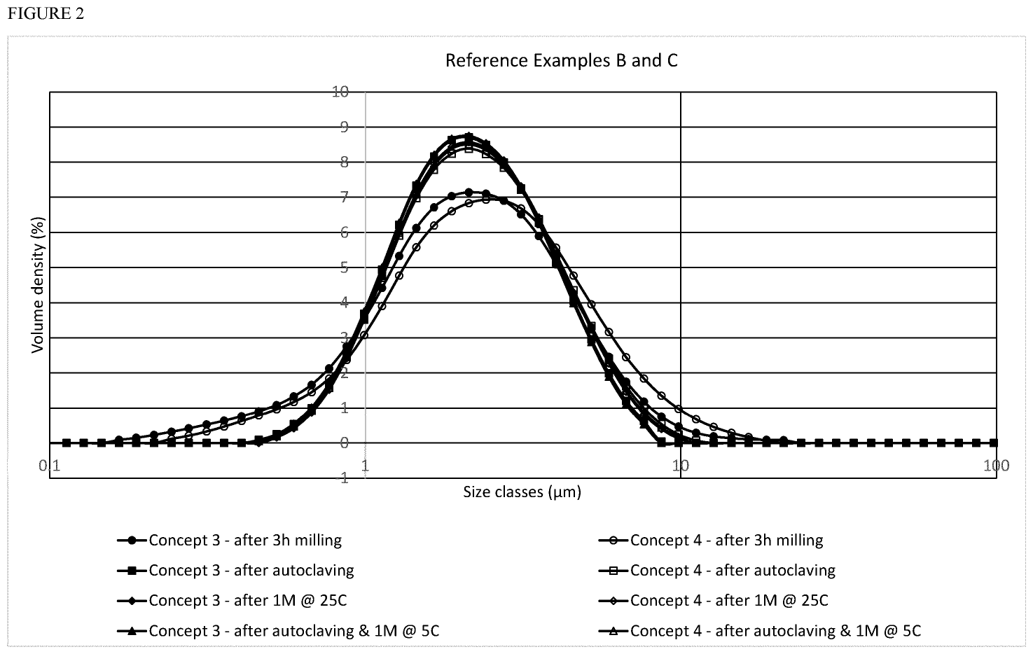

FIG. 2: PSD measurements for Reference Examples B and C under various conditions (including after autoclaving), and where Concept 3 refers to Reference Example B and Concept 4 refers to Reference Example C

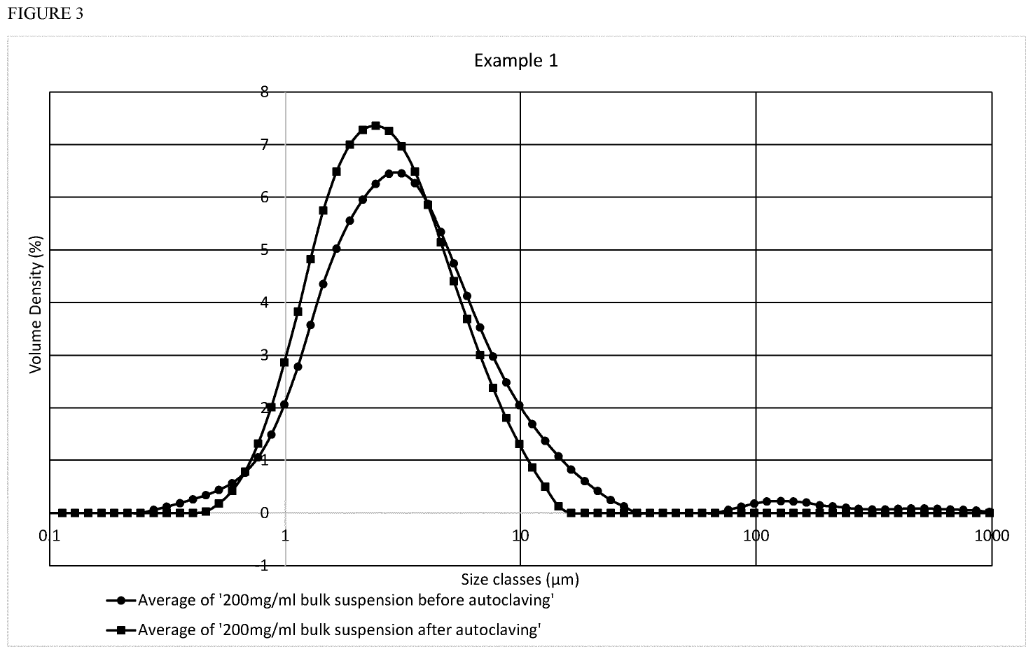

FIG. 3: PSD of the micro-suspension of Example 1, before and after autoclaving

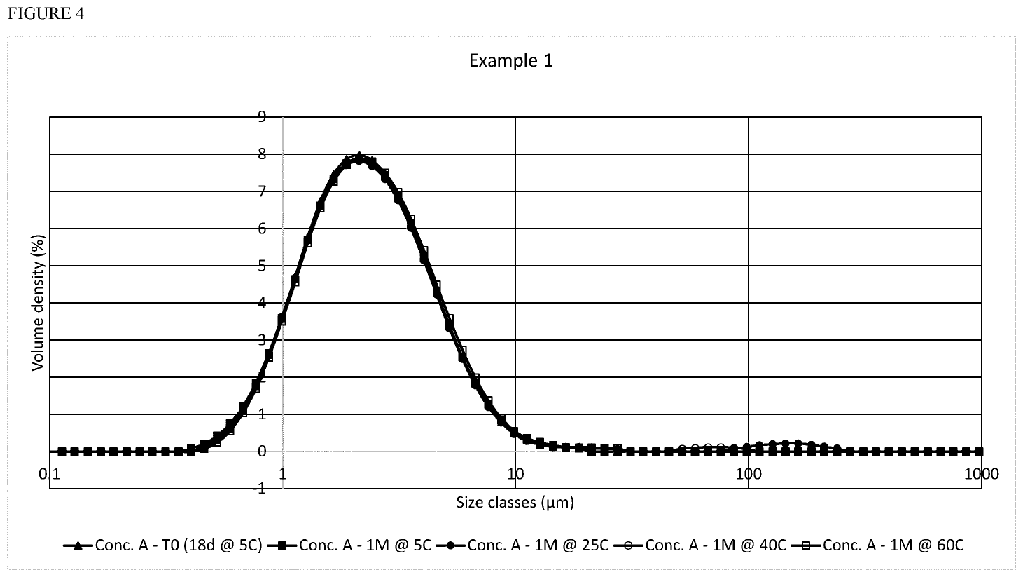

FIG. 4: PSD of the micro-suspension of Example 1, under various conditions including after autoclaving and after further time (and at varying temperatures)

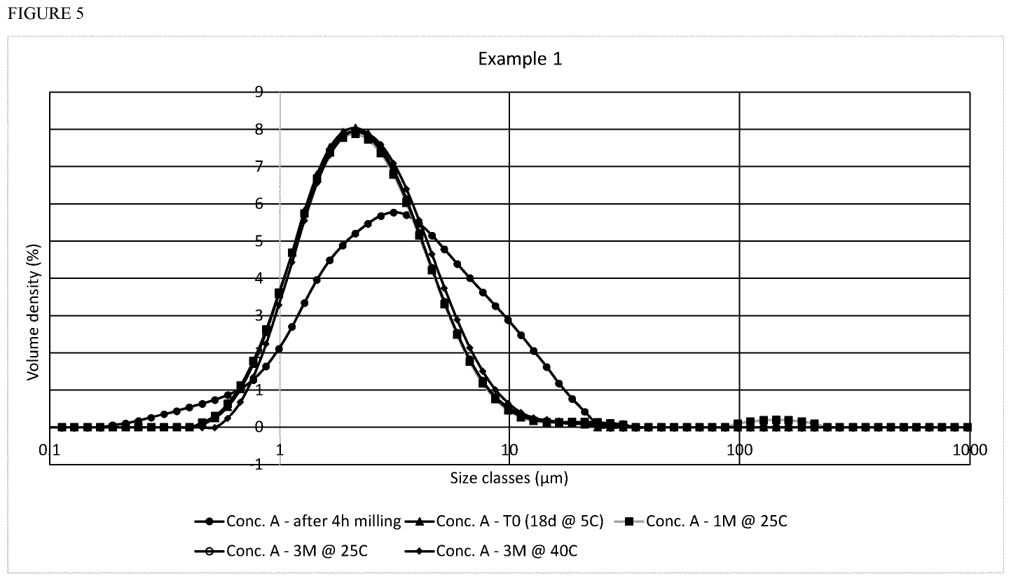

FIG. 5: PSD of the micro-suspension of Example 1, under various other conditions, including up to 3 months at 60° C.

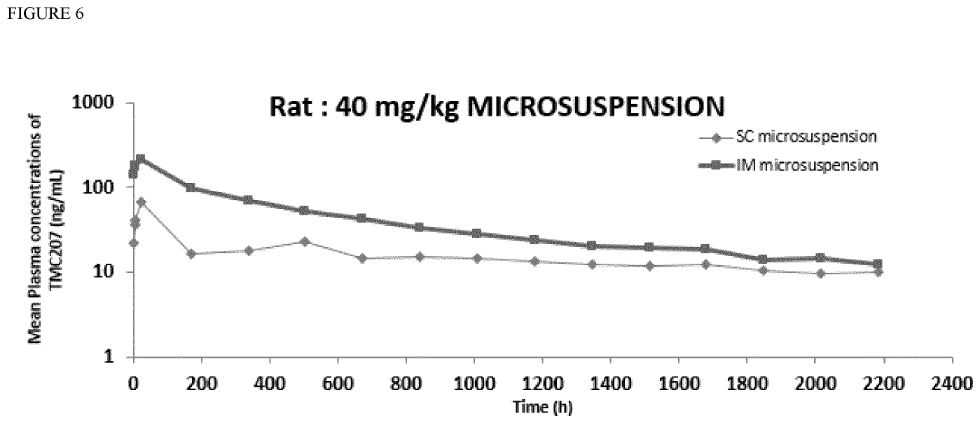

FIG. 6: “Plasma kinetics of TMC207 in male rats when administered IM or SC with 200 mg/ml micro-formulation (see Example 1, Formulation 1B i.e. the micro-suspension) at a dose of 40 mg/kg” and “Plasma kinetics of TMC207 in male rats when administered IM or SC with 200 mg/ml nano-formulation (see Example 1, Formulation 1A, i.e. the nano-suspension) at a dose of 40 mg/kg”

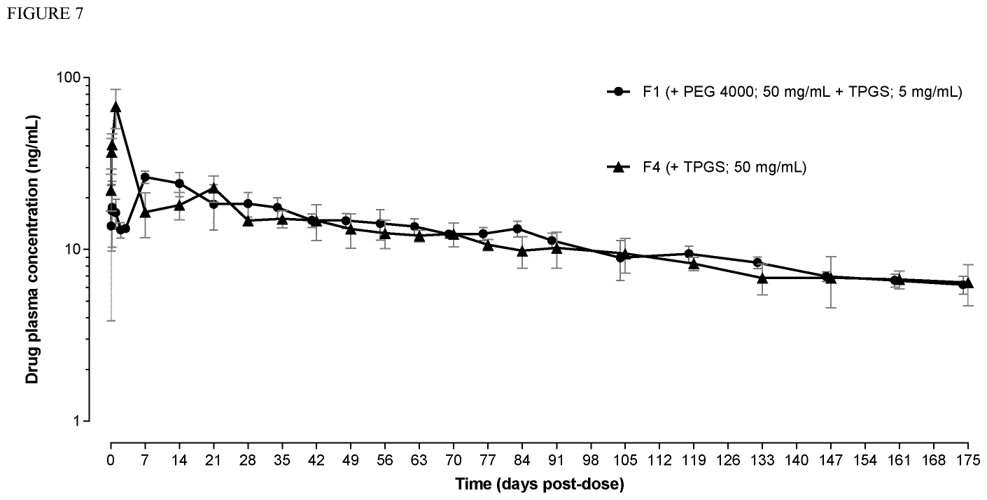

FIG. 7: Plasma concentration versus time profiles of subcutaneous administered bedaquiline LAI micro-suspensions containing different surfactants (PEG4000 combined with TPGS, and TPGS) in rats; data represent means with SD

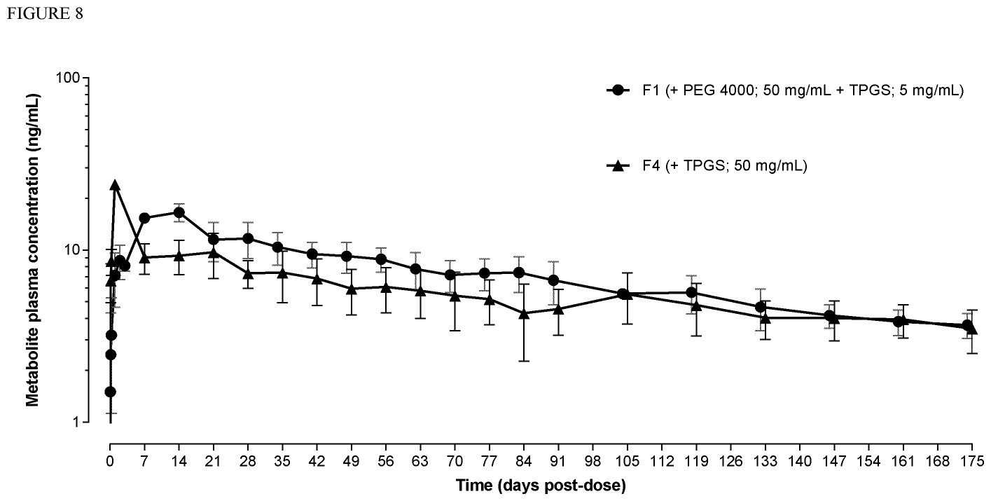

FIG. 8: Plasma concentration versus time profiles of bedaquiline (BDQ) metabolite after subcutaneous administration of BDQ LAI micro-suspensions containing different surfactants (PEG4000 combined with TPGS, and TPGS) in rats; data represent means with SD

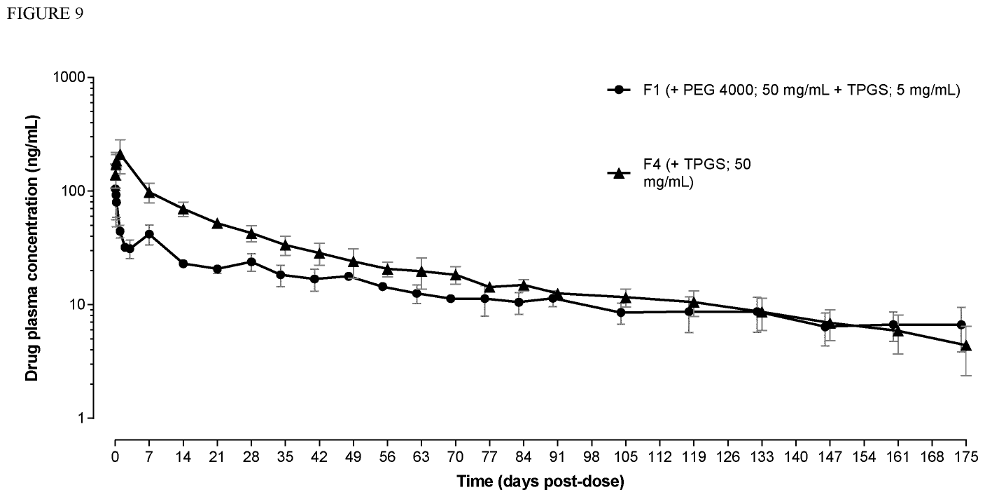

FIG. 9: Plasma concentration versus time profiles of intramuscular administered bedaquiline LAI micro-suspensions containing different surfactants (PEG4000 combined with TPGS, and TPGS) in rats; data represent means with SD

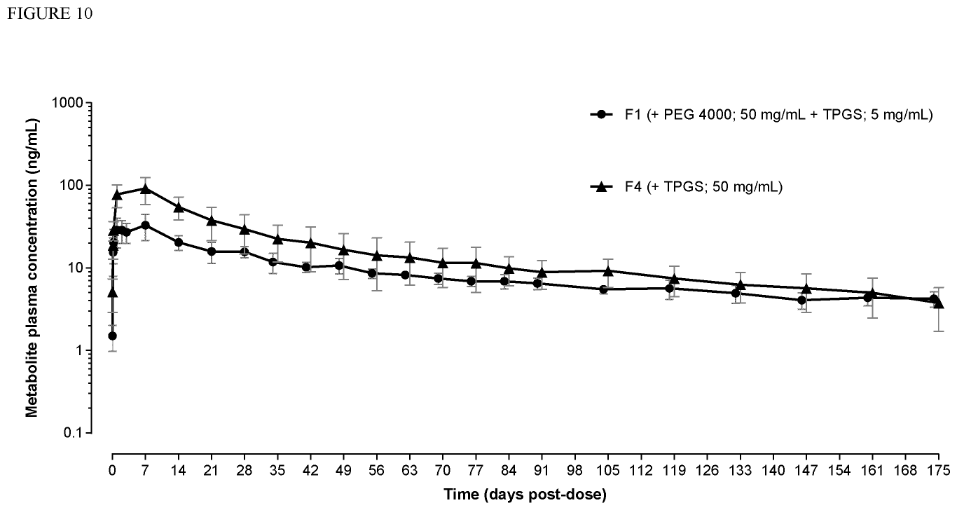

FIG. 10: Plasma concentration versus time profiles of bedaquiline (BDQ) metabolite after intramuscular administration of BDQ LAI micro-suspensions containing different surfactants (PEG4000 combined with TPGS, and TPGS) in rats; data represent means with SD

### DETAILED DESCRIPTION OF THE INVENTION

The compositions of the invention may contain any suitable active pharmaceutical ingredient, as hereinbefore described. However, in an embodiment, the pharmaceutical ingredient is a suitable antibiotic, antibacterial (e.g. anti-tuberculosis) or antiviral drug. In a specific embodiment, the compound used in the invention is the compound TMC207, also referred to as bedaquiline.

Bedaquiline can be used in its non-salt form or as a suitable pharmaceutically acceptable salt form, such as an acid addition salt form or base addition salt form. In an embodiment, bedaquiline is in its non-salt form in compositions of the invention.

The pharmaceutically acceptable acid addition salts are defined to comprise the therapeutically active non-toxic acid addition salt forms which bedaquiline is able to form. Said acid addition salts can be obtained by treating the free form of bedaquiline with appropriate acids, for example inorganic acids, for example hydrohalic acid, in particular hydrochloric acid, hydrobromic acid, sulfuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicyclic acid, p-aminosalicylic acid and pamoic acid. In particular, the fumarate salt is considered, given that this is the form employed in the already-marketed product Sirturo®.

Possible therapeutically active non-toxic base addition salt forms may be prepared by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said acid or base addition salt forms can be converted into the free forms by treatment with an appropriate base or acid.

The term addition salt as used in the framework of this application also comprises the solvates which bedaquiline as well as the salts thereof, are able to form. Such solvates are, for example, hydrates and alcoholates.

Whenever reference to bedaquiline (or TMC207) is employed herein, we refer to the single stereoisomeric form that is employed in the marketed product Sirturo®, and which is disclosed in WO2004/011436 as an antimycobacterial agent.

It has been found that the physico-chemical properties of bedaquiline allow for the manufacture of micro- or nanoparticle suspensions that have unique pharmacokinetic properties in that they can be used for the long term treatment of a pathogenic mycobacterial infection as well as in the long term prevention of a pathogenic mycobacterial infection and to this purpose only a limited number of drug administrations is required. This is beneficial in terms of pill-burden as well as patient compliance with the prescribed dose regimen.

As used herein the term “treatment of a pathogenic mycobacterial infection” relates to the treatment of a subject being infected with a pathogenic mycobacterial infection. Such mycobacterial infection may be Mycobacterium tuberculosis or multi-drug resistance Mycobacterium tuberculosis.

The term “prevention of a pathogenic mycobacterial infection” relates to the prevention or avoidance of a subject becoming infected with a pathogenic mycobacterial infection. The source of infection can be various, for instance a material containing a pathogenic mycobacterial infection.

The terms “therapeutically effective amount”, “an amount, effective in preventing a pathogenic mycobacterial infection”, and similar terms, refer to amounts, or concentrations, of the compositions of the invention (or amounts/concentrations of active ingredient bedaquiline within such compositions) that result in efficacious plasma levels. With “efficacious plasma levels” it is meant those plasma levels of bedaquiline that provide effective treatment or effective prevention of a pathogenic mycobacterial infection. This is because amount/dose/administration given may be linked to the desired exposure levels or desired plasma levels for the effective treatment/prevention, for instance as described herein (see e.g. the examples).

The term “subject” in particular relates to a human being.

The term “micro- or nano-particles” refers to particles in the micrometer or nanometer range. The size of the particles should be below a maximum size above which administration by subcutaneous or intramuscular injection becomes impaired or is even no longer possible. Said maximum size depends for example on the limitations imposed by the needle diameter or by adverse reactions of the body to large particles, or both. In one embodiment, the pharmaceutical compositions of the invention comprise active ingredient (e.g. bedaquiline) in microparticle form. In another embodiment, the pharmaceutical compositions of the invention comprise active ingredient (e.g. bedaquiline) in nanoparticle form.

The average effective particle size of the micro- or nano-particles of the present invention may be below about 50 μm, or below about 20 μm, or below about 10 μm, or below about 1000 nm, or below about 500 nm, or below about 400 nm, or below about 300 nm, or below about 200 nm. The lower limit of the average effective particle size may be low, e.g. as low as about 100 nm or as low as about 50 nm. In one embodiment, the average effective particle size is in the range of about 50 nm to about 50 μm, or about 50 nm to about 20 μm, or about 50 nm to about 10 μm, or about 50 nm to about 1000 nm, about 50 nm to about 500 nm, or about 50 nm to about 400 nm, or about 50 nm to about 300 nm, or about 50 nm to about 250 nm, or about 100 nm to about 250 nm, or about 150 nm to about 220 nm, or 100 to 200 nm, or about 150 nm to about 200 nm, e.g. about 130 nm, or about 150 nm. For instance, both after preparation and after a period of time of up to 3 months (e.g. when stored at temperatures of about 5° C., 25° C. and 40° C.) generally:
the micro-suspensions may have, in an embodiment, a D90 of between about 3 and 10 μm (e.g. about 3.5, 4 or 5 μm) and a D50 of between about 2 and 4 μm (e.g. about 3 μm)
the nano-suspensions may have, in an embodiment, a D90 of between about 0.5 and 1.5 μm (e.g. about, or less than 1 μm or about, or less than about 1000 nm) and a D50 of between about 0.1 and 0.5 μm (e.g. about, or less than, about 0.3 μm, or less than about 300 nm).

In an embodiment, the micro-particles are employed, wherein the average effective particle size, as measured by D10, D50 and/or D90 (in an embodiment as measured by D50) is below about 50 μm, or below about 20 μm, and above about 0.1 μm (100 nm). In an embodiment the range for such micro-particles employed in the compositions of the invention is between about 20 μm and about 0.1 μm (in a further embodiment between about 15 μm, and above about 0.2 μm (200 nm) and in a further embodiment between about 10 μm, and above 0.5 μm (500 nm), for instance between about 10 μm, and above 1 μm or above about 1000 nm, or above about 500 nm, or above about 400 nm, or above about 300 nm, or above about 200 nm. The foregoing values refer to measurements after preparation. They may also, however, in an embodiment, refer to measurements after a period of time up to 3 months (e.g. after 5 days, one week, two weeks, one month, two months or three months) and stored at various temperatures (e.g. at temperatures of about 5° C., 25° C. and 40° C.).

As used herein, the term average effective particle size has its conventional meaning as known to the person skilled in the art and can be measured by art-known particle size measuring techniques such as, for example, sedimentation field flow fractionation, photon correlation spectroscopy, laser diffraction or disk centrifugation. The average effective particle sizes mentioned herein may be related to volume distributions of the particles. In that instance, by “an effective average particle size of less than about 50 μm” it is meant that at least 50% of the volume of the particles has a particle size of less than the effective average of 50 μm, and the same applies to the other effective particle sizes mentioned. In a similar manner, the average effective particle sizes may be related to weight distributions of the particles but usually this will result in the same or about the same value for the average effective particle size.

The pharmaceutical compositions of the present invention provide release of the active ingredient (e.g. bedaquiline) over a prolonged period of time and therefore they can also be referred to as sustained or delayed release compositions. After administration, the compositions of the invention stay in the body and steadily release active ingredient (e.g. bedaquiline), keeping such levels of this active ingredient in the patient's system for a prolonged period of time, thereby providing, during said period, the appropriate treatment or prevention of a pathogenic mycobacterial infection. Because of the fact that the pharmaceutical compositions of the invention stay in the body and steadily release active ingredient, e.g. bedaquiline (and its active metabolite, referred to as M2 herein; see hereinafter, the methyl-substituted metabolite), they can be referred to as pharmaceutical compositions suitable as long-acting (or depot) formulations.

As used herein with the term “prolonged period of time”, there is meant a term (or time period) that may be in the range of one week up to one year or up to two years, or a term in the range of one to two weeks, or two to three weeks, or three to four weeks, or a term in the range of one to two months, or two to three months, or three to four months, or three to six months, or six months to 12 months, or 12 months to 24 months, or a term that is in the range of several days, e.g. 7, 10 or 12 days, or several weeks, e.g. 2, 3 or 4 weeks, or one month, or several months, e.g. 2, 3, 4, 5 or six months or even longer, e.g. 7, 8, 9 or 12 months.

The pharmaceutical compositions of this invention may be applied in the long-term treatment or the long-term prevention of a disease or condition, such as a disease or condition associated with a bacterial or viral infection, e.g. pathogenic mycobacterial infection, or with other words they may be used in the treatment of a pathogenic mycobacterial infection, or in the prevention of a pathogenic mycobacterial infection, during a prolonged period of time. The compositions of the invention are effective in the treatment or prevention of a disease or condition, such as a pathogenic mycobacterial infection for a prolonged period of time, for example for at least about one week or longer, or for about 1 month or longer. By the expression “effective for at least about one week or longer”, one means that the plasma level of the active ingredient, e.g. bedaquiline (and/or its active metabolite M2), should be above a threshold value. In case of therapeutic application said threshold value is the lowest plasma level at which the active ingredient, e.g. bedaquiline (and/or its active metabolite M2), provides effective treatment of a pathogenic mycobacterial infection. In case of application in the prevention of a pathogenic mycobacterial infection said threshold value is the lowest plasma level at which the active ingredient, e.g. bedaquiline (and/or its active metabolite M2), is effective in preventing transmission of a pathogenic mycobacterial infection.

With “long term” for example as used in relation to “long term prevention of a pathogenic mycobacterial infection” or “long term treatment of a pathogenic mycobacterial infection”, or similar terminology, there are meant terms that may be in the range of one week up to one year or up to two years, or longer, such as five or 10 years. In particular in the case of treatment of a pathogenic mycobacterial infection, such terms will be long, in the order of one to several months, one year or longer. Such terms may also be relatively short, in particular in the case of prevention. Shorter terms are those of several days, e.g. 7, 10 or 12 days, or several weeks, e.g. 2, 3 or 4 weeks, or one month, or several months, e.g. 2, 3, 4, 5 or six months or even longer, e.g. 7, 8, 9 or 12 months. In one embodiment the methods and uses in accordance with the present invention are for the prevention of a pathogenic mycobacterial infection during one month, or several months, e.g. 2, 3, 4, 5 or six months or even longer, e.g. 7, 8, 9 or 12 months.

The pharmaceutical compositions of the present invention can be administered at various time intervals. When used in the prevention of a pathogenic mycobacterial infection, the pharmaceutical compositions of this invention can be administered only once or a limited number of times such as twice, three, four, five or six times, or more. This may be recommendable where prevention is required during a limited period of time, such as the period during which there is a risk of infection.

The pharmaceutical compositions of the present invention can be administered at the time intervals mentioned above, such as at a time interval that is in the range of one week to one month, or in the range of one month to three months, or in the range of three months to six months, or in the range of six months to twelve months. In one embodiment, the pharmaceutical composition can be administered once every two weeks, or once every month, or once every three months. In another embodiment the time interval is in the range of one to two weeks, or two to three weeks, or three to four weeks, or the time interval is in the range of one to two months, or two to three months, or three to four months, or three to six months, or six months to 12 months, or 12 months to 24 months. The time interval may be at least one week, but may also be several weeks, e.g. 2, 3, 4, 5 or 6 weeks, or at time intervals of one month, or of several months, e.g. 2, 3, 4, 5 or 6 months or even longer, e.g. 7, 8, 9 or 12 months. In one embodiment, the pharmaceutical compositions of the present invention are administered at a time interval of one, two or three months. These longer periods between each administration of the pharmaceutical compositions of the invention provide further improvements in terms of pill burden and compliance. To further improve compliance, patients can be instructed to take their medication at a certain day of the week, where the composition is administered on a weekly schedule, or at a certain day of the month in case of a monthly schedule.

The length of the time intervals between each administration of a composition of the present invention may vary. For example said time intervals may be selected in function of the plasma levels. The intervals may be shorter where the plasma levels of active ingredient, e.g. bedaquiline (and/or its active metabolite M2), are deemed too low, e.g. when these approach the minimum plasma level specified hereinafter. The intervals may be longer where the plasma levels of active ingredient, e.g. bedaquiline (and/or its active metabolite M2), are deemed too high. In one embodiment, the compositions of the invention are administered at equal time intervals. The compositions may be administered without any interjacent additional administrations, or with other words, the compositions may be administered at particular points in time separated from one another by a time period of varying or equal length, e.g. a time period of at least one week, or any other time period specified herein, during which no further active ingredient, e.g. bedaquiline, is administered. Having time intervals of the same length has the advantage that the administration schedule is simple, e.g. administration takes place at the same day in the week, or the same day in the month. Such administration schedule therefore involves limited “pill burden” thereby contributing beneficially to the patient's compliance to the prescribed dosing regimen.

The concentration (or “C”) of active ingredient, e.g. bedaquiline (and/or its active metabolite M2), in the plasma of a subject treated therewith is generally expressed as mass per unit volume, typically nanograms per milliliter (ng/ml). For convenience, this concentration may be referred to herein as “plasma drug concentration” or “plasma concentration”.

The dose (or amount) of active ingredient, e.g. bedaquiline, administered, depends on the amount of active ingredient, e.g. bedaquiline, in the pharmaceutical compositions of the invention, or on the amount of a given composition that is administered. Where higher plasma levels are desired, either or both of a composition of higher active ingredient, e.g. bedaquiline, concentration, or more of a given composition, may be administered. This applies vice versa if lower plasma levels are desired. Also a combination of varying time intervals and varying dosing may be selected to attain certain desired plasma levels.

The dose (or amount) of active ingredient, e.g. bedaquiline, administered also depends on the frequency of the administrations (i.e. the time interval between each administration). Usually, the dose will be higher where administrations are less frequent. All these parameters can be used to direct the plasma levels to desired values

The dosing regimen also depends on whether prevention or treatment of the disease or condition, e.g. pathogenic mycobacterial infection is envisaged. In case of therapy, the dose of active ingredient, e.g. bedaquiline, administered or the frequency of dosing, or both, are selected so that the plasma concentration of active ingredient, e.g. bedaquiline, is kept above a minimum plasma level. The term “minimum plasma level” (or Cₘᵢₙ) in this context refers to the plasma level of active ingredient, e.g. bedaquiline (and/or its active metabolite M2), that provides effective treatment of the pathogenic mycobacterial infection. In particular, the plasma level of active ingredient, e.g. bedaquiline (and/or its active metabolite M2), is kept at a level above a minimum plasma level of about 10 ng/ml, or above about 15 ng/ml, or above about 20 ng/ml, or above about 40 ng/ml. The plasma level of active ingredient, e.g. bedaquiline (and/or its active metabolite M2), may be kept above a minimum plasma level that is higher, for example above about 50 ng/ml, or above about 90 ng/ml, or above about 270 ng/ml, or above about 540 ng/ml. In one embodiment, the plasma level of active ingredient, e.g. bedaquiline (and/or its active metabolite M2), is kept above a level of about 13.5 ng/ml, or is kept above a level of about 20 ng/ml. Or the plasma level of active in ingredient, e.g. bedaquiline (and/or its active metabolite M2), may be kept within certain ranges, in particular ranges starting from a minimum plasma level selected from those mentioned above and ending at a higher plasma levels selected from those mentioned above and selected from 500 ng/ml and 1000 ng/ml (e.g. from 10 to 15, 10 to 20, 10 to 40, etc., or from 15 to 20, or 15 to 40, or 15 to 90, etc., or 20 to 40, 20 to 90, or 20 to 270, etc., or 40 to 90, 40 to 270, or 40−540, etc., each time from about the indicated value in ng/ml to about the indicated value in ng/ml). In one embodiment said range is from about 10 to about 20, from about 20 to about 90, from 90 to 270, from 270 to 540, from 540 to 1000, each time from about the indicated value in ng/ml to about the indicated value in ng/ml.

The plasma levels of active ingredient, e.g. bedaquiline (and/or its active metabolite M2), should be kept above the above-mentioned minimum plasma levels because at lower levels the bacteria may no longer be sufficiently suppressed so that it can multiply with the additional risk of the emergence of mutations.

In the instance of prevention, the term “minimum plasma level” (or Cₘᵢₙ) refers to the lowest plasma level of active ingredient, bedaquiline (and/or its active metabolite M2), that provides effective treatment/prevention of infection.

In particular, in the instance of prevention, the plasma level of active ingredient, e.g. bedaquiline (and/or its active metabolite M2), can be kept at a level above a minimum plasma level mentioned above in relation to therapy. However in prevention the plasma level of active ingredient, e.g. bedaquiline (and/or its active metabolite M2), can be kept at a lower level, for example at a level above about 4 ng/ml, or about 5 ng/ml, or about 8 ng/ml. The plasma levels of active ingredient, e.g. bedaquiline (and/or its active metabolite M2), should preferably be kept above these minimum plasma levels because at lower levels the drug may no longer be effective thereby increasing the risk of transmission of infection. Plasma levels of active ingredient, e.g. bedaquiline (and/or its active metabolite M2), may be kept at somewhat higher levels to have a safety margin. Such higher levels start from about 50 ng/ml or more. The plasma level of active ingredient, e.g. bedaquiline (and/or its active metabolite M2), can be kept at a level that is in the ranges mentioned above in relation to therapy, but where the lower limits include the plasma levels of about 4 ng/ml, or about 5 ng/ml, or about 8 ng/ml.

An advantage of bedaquiline (and/or its active metabolite M2) is that it may be used up to relatively high plasma levels without any significant side effects. The plasma concentrations of bedaquiline (and/or its active metabolite M2) may reach relatively high levels, but as with any drug should not exceed a maximum plasma level (or Cₘₐₓ), which is the plasma level where bedaquiline (and/or its active metabolite M2) causes significant side effects. Additionally, compound-release from the tissue should also be taken into account, which is not counted for within plasma levels. As used herein, the term “significant side effects” means that the side effects are present in a relevant patient population to an extend that the side effects affect the patients' normal functioning. In an embodiment, the amount and the frequency of administrations of bedaquiline (and/or its active metabolite M2) to be administered are selected such that the plasma concentrations are kept during a long term at a level comprised between a maximum plasma level (or Cₘₐₓ as specified above) and a minimum plasma level (or Cₘᵢₙ as specified above).

In certain instances it may be desirable to keep the plasma levels of active ingredient, e.g. bedaquiline (and/or its active metabolite M2), at relatively low levels, e.g. as close as possible to the minimum plasma levels specified herein. This will allow reducing the frequency of the administrations and/or the quantity of active ingredient, e.g. bedaquiline (and/or its active metabolite M2), administered with each administration. It will also allow avoiding undesirable side effects, which will contribute to the acceptance of the dosage forms in most of the targeted population groups who are healthy people at risk of being infected and therefore are less inclined to tolerate side effects. The plasma levels of active ingredient, e.g. bedaquiline (and/or its active metabolite M2), may be kept at relatively low levels in the instance of prevention. One embodiment concerns uses or methods for prevention of infection, as specified above or hereinafter, wherein the minimum plasma level of active ingredient, e.g. bedaquiline (and/or its active metabolite M2), is as specified herein and the maximum plasma level is about equal to the lowest plasma level that causes the active ingredient to act therapeutically, also as specified herein.

In other embodiments, the plasma level of active ingredient, e.g. bedaquiline (and/or its active metabolite M2), is kept at a level below a lower maximum plasma level of about 10 ng/ml, more in particular about 15 ng/ml, further in particular about 20 ng/ml, still more in particular about 40 ng/ml. In a particular embodiment, the plasma level of active ingredient, e.g. bedaquiline (and/or its active metabolite M2), is kept below a level of about 13.5 ng/ml. In one embodiment, the plasma level of active ingredient, e.g. bedaquiline (and/or its active metabolite M2), is kept in an interval of the lower maximum blood level specified above, and the minimum plasma levels mentioned in relation to prevention. For example the plasma levels of active ingredient, e.g. bedaquiline (and/or its active metabolite M2), are kept below about 10 ng/ml and above a minimum level of about 4 ng/ml.

In other instances it may be desirable to keep the plasma levels of active ingredient, e.g. bedaquiline (and/or its active metabolite M2), at relatively higher levels, for example where there is a high risk of infection and more frequent and/or higher doses are not an issue. In these instances the minimum plasma level may be equal to the lowest plasma level of active ingredient, e.g. bedaquiline (and/or its active metabolite M2), that provides effective treatment of a pathogenic mycobacterial infection, such as the specific levels mentioned herein.

In the instance of prevention, the dose to be administered should be calculated on a basis of about 0.2 mg/day to about 50 mg/day, or 0.5 mg/day to about 50 mg/day, or of about 1 mg/day to about 10 mg/day, or about 2 mg/day to about 5 mg/day, e.g. about 3 mg/day. This corresponds to a weekly dose of about 1.5 mg to about 350 mg, in particular of about 3.5 mg to about 350 mg, in particular of about 7 mg to about 70 mg, or about 14 mg to about 35 mg, e.g. about 35 mg, or to a monthly dose of from 6 mg to about 3000 mg, in particular about 15 mg to about 1,500 mg, more in particular of about 30 mg to about 300 mg, or about 60 mg to about 150 mg, e.g. about 150 mg. Doses for other dosing regimens can readily be calculated by multiplying the daily dose with the number of days between each administration.

In the instance of therapy, the dose to be administered should be somewhat higher and should be calculated on a basis of about 1 mg/day to about 150 mg/day, or of about 2 mg/day to about 100 mg/day, or of about 5 mg/day to about 50 mg/day, or about 10 mg/day to about 25 mg/day, e.g. about 15 mg/day. The corresponding weekly or monthly doses can be calculated as set forth above. For applications in prevention, the doses may be lower although the same dosing as for therapeutic applications may be used. In an embodiment, the dose/administration is given at monthly intervals or three-monthly or six-monthly intervals, with the total treatment duration being three, six or 12 months. In the instances where the dose/administration is monthly, three monthly or six-monthly, in an embodiment, the dose given (e.g. in human subjects) is calculated on the basis of a 400 mg daily dose given for 2 weeks. Hence, the total amount of active ingredient, e.g. bedaquiline, given per dose may be about 5600 mg (e.g. in the range of 3000 and 8000 mg), but it may be up to one fifth of such an amount (e.g. in the range of 500 and 2000 mg, e.g. between about 1000 and 1500 mg).

In another embodiment, in the case of prevention or in particular therapy, the doses may also be expressed in mg/kg. For instance, in the examples, certain doses may be administered based on weight (of e.g. the mammal, and as shown in the examples here, in mouse) and hence doses between 1 mg/kg and 1000 mg/kg may be employed (e.g. 40 mg/kg, 80 mg/kg, 160 mg/kg, 320 mg/kg or 480 mg/kg may be employed) and such doses may remain effective for a period of 4 weeks, 8 weeks or 12 weeks (for example as shown in the examples). For instance, one dose may be taken every 4 weeks (effectively seen as a 12 week treatment regimen, i.e. three doses in total) or one single dose may be taken, which effectively provides sufficient treatment (e.g. as defined by reduction in CFUs, see examples) as may be evidenced by monitoring over a 12 week period. Hence, in an aspect, in order to treat the bacterial infection one dose may be taken (e.g. between 1 mg/kg and 1000 mg/kg, for instance between 2 mg/kg and 500 mg/kg) or one such dose may be taken every 4 weeks (e.g. two or three such doses may be taken). Such dose depends on the bacterial infection to be treated. For instance, in the treatment of latent tuberculosis or leprosy, lower doses may be required (compared to e.g. multi-drug resistant tuberculosis) given that a lower amount of bedaquiline is required to control the bacteria. An example of this is described hereinafter, wherein it is indicated that in mice one dose of 160 mg/kg may sufficiently reduce CFUs in the mouse model of latent tuberculosis infection—it was also seen that two or three doses of 160 mg/kg (the second and the third doses administered at 4 and 8 weeks, respectively) were also effective in that model.

It has been found that, once administered, the plasma levels of active ingredient, e.g. bedaquiline (and/or its active metabolite M2), are more or less stable, i.e. they fluctuate within limited margins. The plasma levels have been found to approach more or less a steady state mode or to approximate more or less a zero order release rate during a prolonged period of time. By “steady state” is meant the condition in which the amount of drug present in the plasma of a subject stays at more or less the same level over a prolonged period of time. The plasma levels of active ingredient, e.g. bedaquiline (and/or its active metabolite M2), generally do not show any drops below the minimum plasma level at which the drug is effective. The term “stays at more or less the same level” does not exclude that there can be small fluctuations of the plasma concentrations within an acceptable range, e.g. fluctuations within a range of about ±30%, or about ±20%, or about ±10%, or about ±10%.

In some instances there may be an initial plasma concentration peak after administration, after which the plasma levels achieve a “steady-state”, as mentioned hereinafter.

The compositions of the invention show good local tolerance and ease of administration. Good local tolerance relates to minimal irritation and inflammation at the site of injection; ease of administration refers to the size of needle and length of time required to administer a dose of a particular drug formulation. In addition, the compositions of the invention show good stability and have an acceptable shelf life.

The micro- or nano-particles of the present invention have a surface modifier adsorbed on the surface thereof. The function of the surface modifier is to act as a wetting agent as well as a stabilizer of the colloidal suspension.

In one embodiment, the micro- or nano-particles in the compositions of the invention mainly comprise crystalline active ingredient (e.g. bedaquiline or a salt thereof); and a surface modifier, the combined amount of which may at least comprise about 50%, or at least about 80%, or at least about 90%, or at least about 95%, or at least about 99% of the micro- or nano particles. As indicated herein, in an embodiment, bedaquiline is in its non-salt form (or in its “free form”) and in a further embodiment it is in a crystalline non-salt (or free) form. In this respect, as mentioned herein, bedaquiline may be prepared as such using the procedures described in international patent application WO 2004/011436 (or in WO 2006/125769, which describes an optical resolution with a chiral reagent). Following such procedure, the bedaquiline is obtained by precipitation from toluene/ethanol and it is indicated that the product crystallises. Such form of bedaquiline may be used in the preparation of the compositions of the invention and, further, such form may be a single crystalline polymorph with the following characterising features:
(i) a melting endotherm at 181.5° C. (endotherm onset) and DSC curve showing melting of the product at about 182.5° C. (immediately followed by decomposition; measured by differential scanning calorimetry (DSC) by transfer of about 3 mg of compound into a standard aluminum TA-Instrument sample pan, sample pan closed with the appropriate coer and DSC curve recorded on a TA-Instruments Q2000 MTDSC equipped with a RCS cooling unit using the following parameters—initial temperature 25° C.; heating range 10° C./min; final temperature 300° C., nitrogen flow 50 ml/min);
(ii) infrared (IR) spectrum peaks at inter alia about 1600 cm⁻¹, about 1450 cm⁻¹, about 1400 cm⁻¹, about 1340 cm⁻¹, and about 1250 cm⁻¹ (where a sample is analysed using a suitable microATR accessory deploying 32 scans, 1 cm⁻¹ resolution, Thermo Nexus 670 FTIR spectrometer, a DTGS with KBr windows detector, Ge on KBr beamsplitter and a micro ATR accessory (Harrick Split Pea with Si crystal); and/or
(iii) X-ray powder diffraction (XRPD) with characteristic peaks at about 11.25° 2-Theta, about 18° 2-Theta, about 18.5° 2-Theta, about 19° 2-Theta, about 20.25° 2-Theta, about 21.25° 2-Theta, about 22.25° 2-Theta, about 24.5° 2-Theta and about 27° 2-Theta, showing diffraction peaks without the presence of a halo indicating crystallinity of the product (where the analysis was carried out on a PANalytical (Philips) X'PertPRO MPD diffractometer, and the instrument is equipped with a Cu LFF X-ray tube and the compound was spread on a zero background sample holder; the Instrument Parameters were: generator voltage—45 kV; generator amperage—40 mA; geometry—Bragg-Brentano; stage—spinner stage; scan mode—continuous; scan range 3 to 50° 2θ; step size 0.02°/step; counting time 30 sec/step; spinner revolution time—1 sec; radiation type CuKα).

Hence, in an embodiment, the active ingredient, e.g. bedaquiline, employed in a process to prepare compositions of the invention (i.e. before conversion to micro/nano-particles) is a crystalline form (e.g. of the specific form characterised above). In a further embodiment of the invention, the active ingredient, e.g. bedaquiline, employed in the compositions of the invention (i.e. after conversion to micro/nano-particles, for instance by milling) is also in a crystalline form (e.g. of the specific form characterised above).

In a further aspect, the present invention is concerned with a pharmaceutical composition for administration by intramuscular or subcutaneous injection, comprising a therapeutically effective amount of an active pharmaceutical ingredient, or a pharmaceutically acceptable salt thereof, in the form of a suspension of particles consisting essentially of:
(1) an active pharmaceutical ingredient, or a pharmaceutically acceptable salt thereof in micro- or nanoparticle form, having a surface modifier adsorbed to the surface thereof; and
(2) a pharmaceutically acceptable aqueous carrier; wherein the active ingredient is suspended,
   which is characterised in that the surface modifier comprises PEG4000 or the like, and in which the composition undergoes sterilization (e.g. by autoclaving) and re-suspension.

It is indicated that the formulations of the invention contain PEG4000 (or the like), and for the avoidance of doubt, this may be in combination with another suitable surface modifier.

Suitable surface modifiers (that may be used in combination with PEG4000, or the like) can be selected from known organic and inorganic pharmaceutical excipients, including various polymers, low molecular weight oligomers, natural products and surfactants. Particular surface modifiers include nonionic and anionic surfactants. Representative examples of surface modifiers include gelatin, casein, lecithin, salts of negatively charged phospholipids or the acid form thereof (such as phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG), gum acacia, stearic acid, benzalkonium chloride, polyoxyethylene alkyl ethers, e.g., macrogol ethers such as cetomacrogol 1000, polyoxyethylene castor oil derivatives; polyoxyethylene stearates, colloidal silicon dioxide, sodium dodecylsulfate, carboxymethylcellulose sodium, bile salts such as sodium taurocholate, sodium desoxytaurocholate, sodium desoxycholate; methylcellulose, hydroxyethylcellulose, hydroxypropylcellulose, hydroxypropyl-methylcellulose, magnesium aluminate silicate, polyvinyl alcohol (PVA), poloxamers, such as Pluronic™ F68, F108 and F127 which are block copolymers of ethylene oxide and propylene oxide; tyloxapol; Vitamin E-TGPS (α-tocopheryl polyethylene glycol succinate, in particular α-tocopheryl polyethylene glycol 1000 succinate); poloxamines, such as Tetronic™ 908 (T908) which is a tetrafunctional block copolymer derived from sequential addition of ethylene oxide and propylene oxide to ethylenediamine; dextran; lecithin; dioctyl ester of sodium sulfosuccinic acid such as the products sold under the tradename Aerosol OT™ (AOT); sodium lauryl sulfate (Duponol™ P); alkyl aryl polyether sulfonate available under the tradename Triton™ X-200; polyoxyethylene sorbitan fatty acid esters (Tweens™ 20, 40, 60 and 80); sorbitan esters of fatty acids (Span™ 20, 40, 60 and 80 or Arlacel™ 20, 40, 60 and 80); sucrose stearate and sucrose distearate mixtures such as the product available under the tradename Crodesta™ F110 or Crodesta™ SL-40; hexyldecyl trimethyl ammonium chloride (CTAC); polyvinylpyrrolidone (PVP). If desired, two or more surface modifiers can be used in combination (with PEG4000 or the like).

Particular surface modifiers that may be employed in combination with PEG4000 (or the like) are selected from poloxamers, α-tocopheryl polyethylene glycol succinates, polyoxyethylene sorbitan fatty acid esters, and salts of negatively charged phospholipids or the acid form thereof. More in particular the surface modifiers are selected from Pluronica™ F108, Vitamin E TGPS, Tween™ 80, and Lipoid™ EPG (and, in a particular embodiment, it is Vitamin E TPGS). One or more of these surface modifiers may be used. Pluronic™ F108 corresponds to poloxamer 338 and is the polyoxyethylene, polyoxypropylene block copolymer that conforms generally to the formula HO—[CH₂CH₂O]ₓ—[CH(CH₃)CH₂O]_{y}—[CH₂CH₂O]_{z}—H in which the average values of x, y and z are respectively 128, 54 and 128. Other commercial names of poloxamer 338 are Hodag Nonionics 1108-F and Synperonic™ PE/F108. In one embodiment, the surface modifier comprises a combination of a polyoxyethylene sorbitan fatty acid ester and a phosphatidyl glycerol salt (in particular egg phosphatidyl glycerol sodium).

The optimal relative amount of active ingredient, e.g. bedaquiline, in relation to the surface modifier depends on the surface modifier selected, the specific surface area of the suspension which is determined by the average effective particle size and the active ingredient, e.g. bedaquiline, concentration, the critical micelle concentration of the surface modifier if it forms micelles, etc. The relative amount (w/w) of active ingredient, e.g. bedaquiline, to the surface modifier preferably is in the range of 1:2 to about 20:1, in particular in the range of 1:1 to about 10:1, e.g. in the range of 2:1 to about 10:1, for instance about 4:1.

As indicated, the surface modifier contains PEG4000, but may also contain a further surface modifier (for instance, a surface modifier mentioned hereinbefore). In various embodiments of the invention, the compositions of the invention comprise a surface modifier that contains PEG4000 and one or more other surface modifiers in the following w/w ratios:
at least 1:10 of PEG4000:one or more other surface modifiers
between 1:10 and 100:1 (e.g. between about 1:10 and 20:1) of PEG4000:one or more other surface modifiers
about 10:1 PEG4000:one or more other surface modifiers

Hence, when the surface modifier of the compositions of the invention comprises a ratio of at least 1:10 w/w of PEG4000 (or the like):one or more other surface modifiers, then it may contain 5 mg/mL PEG4000 and 50 mg/ml of one or more other surface modifier (e.g. Vitamin E TPGS, also referred to herein as simply “TPGS”). In an embodiment, given that the relative amount of active ingredient, e.g. bedaquiline, to the surface modifier may be between 1:1 and 10:1 (e.g. about 4:1), then the active ingredient, e.g. bedaquiline, may be present in about 200 mg/ml in such instances (which may form a particular injectable formulation or dose). While the compositions of the invention are distinguished as they contain PEG4000 (or the like) and it may be a relatively small amount, in an embodiment, the surface modifier comprises at least 25% by weight, for example at least 50% by weight PEG4000 or the like (and the remainder being one or more other suitable surface modifiers as described herein, for example Vitamin E TPGS). For instance, in an embodiment, the surface modifier of the compositions of the invention comprise a ratio of at least 1:1 w/w of PEG4000 or the like:one or more other suitable surface modifiers. In a further embodiment, the surface modifier comprises at least 75% by weight PEG4000 or the like (and the remainder being one or more other suitable surface modifiers as described herein, for example Vitamin E TPGS). Hence, in an embodiment, the surface modifier of the compositions of the invention comprise a ratio of at least 3:1 w/w of PEG4000 or the like:one or more other suitable surface modifiers. In yet further embodiments, the surface modifier of the compositions of the invention comprise at least 85% by weight PEG4000 or the like or between about 85% and about 95% PEG4000 or the like (and in each case, the remainder is one or more other suitable surface modifier as described herein, e.g. Vitamin E TPGS). Hence, in an embodiment the surface modifier of the compositions of the invention comprise a ratio of at least 8:1 w/w of PEG4000 or the like:one or more other suitable surface modifiers (for instance a ratio of between 8:1 and 12:1 w/w of PEG4000 or the like:one or more other suitable surface modifiers).

The ratios of PEG4000 (and the like) and one or more other surface modifiers may also depend on the other surface modifiers being used; for instance when the one or more other surface modifiers comprises Vitamin E TPGS and/or Tween (a polyoxyethylene polyether sulfonate), the ratios hereinabove may be applicable, and for instance the surface modifier comprises at least 60% by weight PEG4000 and, in an embodiment at least 75%; in the case where the one or more other surface modifiers comprises a poloxamer then the ratio may be between 1:10 to 10:1 (of PEG:one or more other surface modifier), for instance between 1:5 and 5:1 and, in an embodiment between 1:2 and 2:1, and, in an embodiment, the surface modifier in this instance comprises at least 30% PEG4000, for instance, at least 40% (and, in a specific embodiment about 50%). In certain instances, at least 10% PEG4000 is required, but the upper limit may be 60% (e.g. when the one or more other surface modifier is a poloxamer).

As indicated, the compositions of the invention comprise a surface modifier that contain PEG4000 or the like. In an embodiment, the surface modifier may consist essentially of PEG4000 or the like. However, in an alternative embodiment, the surface modifier also contains another suitable surface modifier as described herein.

Where one or more other surface modifiers are employed in compositions of the invention, then those other surface modifiers may, in a particular embodiment, be selected from Vitamin E TPGS or a poloxamer. For instance, the other surface modifier may be Vitamin E TPGS. Hence, as indicated herein, the w/w ratio of active ingredient, e.g. bedaquiline, to surface modifier may be in the range 2:1 to 10:1 (e.g. about 4:1) and hence, when 200 mg/ml of active ingredient, e.g. bedaquiline, is employed (e.g. for a single injectable dose), then that may contain between 100 mg/ml and 20 mg/ml surface modifier. In this instance, and again as indicated, the amount of surface modifier may contain PEG4000 (or the like) and one or more other suitable surface modifiers in a ratio of, for example, at least 3:1 (or at least 75% by weight PEG4000). Hence, when there is 100 mg/ml surface modifier present, then that may consist of at least 75 mg/ml PEG4000 or the like, with any remainder consisting of one or more other suitable surface modifiers (e.g. Vitamin E TPGS) and when there is 20 mg/ml surface modifier then this may consist of at least 15 mg/ml PEG4000 or the like, and any remainder consisting of one or more other suitable surface modifier. As it is indicated hereinbefore that the ratio of active ingredient, e.g. bedaquiline, to surface modifier may be about 4:1, then when there is 200 mg/ml active ingredient, e.g. bedaquiline (e.g. as one injectable dose), then the amount of surface modifier may be between about 35 mg/ml and 60 mg/ml (for instance about 55 mg/ml, in which case the surface modifier may contain about 50 mg/ml PEG4000 or the like, and about 5 mg/ml of one or more other surface modifier, e.g. Vitamin E TPGS).

The compositions of the invention may need to be sterile so that they can be administered to patients. Achieving sterile compositions may be done in a number of ways, including manufacturing such compositions in a sterile process or environment. However, such a method has a number of drawbacks, challenges and is associated with higher costs. A preferred alternative is to undergo sterilization without having to conform to an entire sterile process, and heat sterilization, autoclaving and gamma radiations are sterilization steps that can achieve that. Advantageously, compositions of the invention can be autoclaved, i.e. are autoclavable, and that can be done without substantial degradation or decomposition of the compositions.

Further challenges arise after sterilization, which are linked to desired stability of the long-acting formulation, undesired aggregation of particles of the active pharmaceutical ingredient (API) within that formulation and the desired re-suspendability of the formulation (after sterilization, e.g. autoclaving).

In this case, the compositions of the invention may be sterilized, for instance by heat sterilization, autoclaving or gamma radiation (in an embodiment, the sterilization is performed by autoclaving), even though the cloud point may be below the temperature at which autoclaving takes place. Advantageously, the compositions of the invention may be easily resuspended after sterilization (even if the cloud point is exceeded during the sterilization process, in particular the autoclaving process).

Hence, in a further aspect of the invention, there is provided:
(a) a process for sterilizing the compositions of the invention (for instance, autoclaving the compositions);
(b) followed by re-suspending such compositions of the invention,
   which process may be referred to as a “process of the invention”. The examples show that the PEG4000 may be key in re-suspending. It will be understood that after sterilization (e.g. heat sterilization or autoclaving), there may be some particle aggregation (especially if the sterilization process is performed at a temperature above the cloud point), for instance due to phase separation. Given that the compositions of the invention should essentially be a suspension, then the re-suspending step may be necessary (such re-suspending step may also be performed at a later point in time, e.g. when the suspension is being prepared for its end use). The compositions of the invention start as a suspension, with the active ingredient, e.g. bedaquiline, particles suspended in the pharmaceutically acceptable carrier and the surface modifier (i.e. PEG4000 containing surface modifier as hereinbefore defined) may be adsorbed onto the surface of the active ingredient, e.g. bedaquiline—after autoclaving there may be disassociation between the surface modifier (also referred to herein as wetting agent) and the active ingredient, e.g. bedaquiline, and/or active ingredient (e.g. bedaquiline) particle aggregation. Hence, re-suspending back to the original suspension is essential and may be effected by swirling or shaking the composition of the invention (after sterilization, e.g. autoclaving). The re-suspending (of active ingredient, e.g. bedaquiline, in the carrier) may occur by allowing the surface modifier (i.e. PEG4000 and one or more other suitable surface modifiers) to adsorb onto the surface of the active ingredient, e.g. bedaquiline.

As indicated, the re-suspendability after sterilization (e.g. autoclaving) may be linked to the presence of PEG4000. Additionally or alternatively, the use of PEG4000 as a surface modifier may be advantageous as it may replace a surface modifier that may be as efficient (e.g. with similar properties allowing for suspension and/or re-suspendability after sterilization) but where that surface modifier being replaced may not be tolerated (e.g. in humans) above a certain dose or quantity (e.g. as an injectable). For instance, other surface modifiers such as Vitamin E TPGS may not be tolerated above a certain dose as an injectable in humans and hence would either need to be replaced entirely or the dose/amount reduced.

A micro- or nano-suspension (not containing PEG4000) may be sterilized by autoclaving and may be adequately re-suspendable (for example, re-suspendable under conditions defined herein, especially by swirling for less than 40 seconds) in which case PEG4000 may not be needed. However, in an embodiment where the re-suspendability is not adequate (for instance, takes longer than 40 seconds), then the use of PEG4000, or the like, in such a micro- or nano-suspension may assist in improving the re-suspendability (i.e. by making it easier, including by reducing the time taken to less than 40 seconds), for instance after autoclaving. The US Pharmacopoeia indicates that suspensions should be re-dispersible in case they settle upon storage, etc, and a goal is to have a suspension in general where the time taken to re-suspend is as short as possible; in this respect, and in an aspect of the invention thus, PEG4000 (or the like) can assist.

Hence, in view of the above, in further embodiments of the invention, there is provided:
PEG4000, or the like, for use as a surface modifier in a pharmaceutical composition for administration by intramuscular or subcutaneous injection, wherein said composition comprises an active pharmaceutical ingredient (e.g. bedaquiline), or a pharmaceutically acceptable salt thereof, in the form of a suspension of micro- or nano-particles, characterised in that the PEG4000 assists in re-suspending said composition for instance after sterilization (e.g. autoclaving)
PEG4000, or the like, for use in re-suspending a pharmaceutical composition comprising an active pharmaceutical ingredient (e.g. bedaquiline), or a pharmaceutically acceptable salt thereof, in the form of a suspension of micro- or nano-particles, for instance wherein said composition has undergone sterilization (e.g. autoclaving)
PEG4000, or the like, for use as a resuspendability aid in a pharmaceutical composition comprising an active pharmaceutical ingredient (e.g. bedaquiline), or a pharmaceutically acceptable salt thereof, in the form of a suspension of micro- or nano-particles, for instance wherein said composition has undergone sterilization (e.g. autoclaving)
PEG4000, or the like, for use to increase (or improve) the resuspendability of a pharmaceutical composition comprising an active pharmaceutical ingredient (e.g. bedaquiline), or a pharmaceutically acceptable salt thereof, in the form of a suspension of micro- or nano-particles, for instance wherein said composition has undergone sterilization (e.g. autoclaving)

In all cases above, the PEG4000, or the like, may be for such uses in pharmaceutical compositions described herein. Resuspendability may in certain circumstances be compared to the pharmaceutical composition without the PEG4000.

In an alternative further embodiments, there is provided:
the use of PEG4000, or the like, as a surface modifier in a pharmaceutical composition comprising an active pharmaceutical ingredient (e.g. bedaquiline), or a pharmaceutically acceptable salt thereof, in the form of a suspension of micro- or nano-particles, wherein the PEG4000 assists in re-suspending said composition, for instance after sterilization (e.g. autoclaving)
the use of PEG4000, or the like, in re-suspending a pharmaceutical composition comprising an active pharmaceutical ingredient (e.g. bedaquiline), or a pharmaceutically acceptable salt thereof, in the form of a suspension of micro- or nano-particles, for instance wherein said composition has undergone sterilization (e.g. autoclaving)
the use of PEG4000, or the like, as a resuspendability aid in a pharmaceutical composition comprising an active pharmaceutical ingredient (e.g. bedaquiline), or a pharmaceutically acceptable salt thereof, in the form of a suspension of micro- or nano-particles, for instance wherein said composition has undergone sterilization (e.g. autoclaving)
the use of PEG4000, or the like, to increase (or improve) the resuspendability of a pharmaceutical composition comprising an active pharmaceutical ingredient (e.g. bedaquiline), or a pharmaceutically acceptable salt thereof, in the form of a suspension of micro- or nano-particles, for instance wherein said composition has undergone sterilization (e.g. autoclaving)

In all cases above, the use of PEG4000, or the like, may be in pharmaceutical compositions described herein. Again, resuspendability may in certain circumstances be compared to the pharmaceutical composition without the PEG4000.

The particles of this invention can be prepared by means of micronization/particle size reduction/nanonization by mechanical means and by controlled precipitation from a supersaturated solution, or by using supercritical fluids such as in the GAS technique (“gas anti-solvent”), or any combination of such techniques. In one embodiment a method is used comprising the steps of dispersing active ingredient, e.g. bedaquiline, in a liquid dispersion medium and applying mechanical means in the presence of grinding media to reduce the particle size of active ingredient, e.g. bedaquiline, to an average effective particle size of less than about 50 μm, in particular less than about 1,000 nm. The particles can be reduced in size in the presence of a surface modifier.

A general procedure for preparing the particles of this invention comprises
(a) obtaining active ingredient, e.g. bedaquiline, in micronized form;
(b) adding the micronized active ingredient, e.g. bedaquiline, to a liquid medium to form a premix/predispersion; and
(c) subjecting the premix to mechanical means in the presence of a grinding medium to reduce the average effective particle size.

In a particular embodiment, there is provided a process for preparing a pharmaceutical composition comprising
(a) obtaining an active pharmaceutical ingredient (e.g. bedaquiline), or a pharmaceutically acceptable salt thereof, in micronized form;
(b) adding the micronized active ingredient, e.g. bedaquiline, or a pharmaceutically acceptable salt thereof, to a liquid medium to form a premix/predispersion, characterised in that the liquid medium contains a surface modifier comprising PEG4000, or the like, as per any one of claims 1, 2, 3 or 4;
(c) subjecting the premix to mechanical means in the presence of a grinding medium to reduce the average effective particle size;
(d) sterilization (e.g. autoclaving); and
(e) re-suspending (e.g. if needed).

In such instances, the re-suspending may be performed by swirling for less than 40 seconds. In a particular embodiment, there is provided the use of PEG4000 in such (a) process(es).

Active ingredient (e.g. bedaquiline) in micronized form is prepared using techniques known in the art. It is preferred that the average effective particle size of the active ingredient, e.g. bedaquiline active agent, in the predispersion be less than about 100 μm as determined by sieve analysis. Where the average effective particle size of the micronized active ingredient, e.g. bedaquiline, is greater than about 100 μm, it is preferred that the particles of the active ingredient, e.g. bedaquiline, compound be reduced in size to less than 100 μm (for example to a size or size range as described herein).

The micronized active ingredient, e.g. bedaquiline, can then be added to a liquid medium in which it is essentially insoluble to form a predispersion. The concentration of active ingredient, e.g. bedaquiline, in the liquid medium (weight by weight percentage) can vary widely and depends on the selected surface modifier and other factors. Suitable concentrations of active ingredient, e.g. bedaquiline, in compositions vary between about 0.1% to about 60%, or between about 1% to about 60%, or between about 10% to about 50%, or between about 10% to about 30%, e.g. about 10%, 20% or 30% (each % in this paragraph relating to w/v).

The premix can be used directly by subjecting it to mechanical means to reduce the effective average effective particle size in the dispersion to less than 2,000 nm. It is preferred that the premix be used directly when a ball mill is used for attrition. Alternatively, active ingredient, e.g. bedaquiline, and, optionally, the surface modifier, can be dispersed in the liquid medium using suitable agitation such as, for example, a roller mill, until a homogeneous dispersion is achieved.

The mechanical means applied to reduce the effective average effective particle size of active ingredient, e.g. bedaquiline, conveniently can take the form of a dispersion mill. Suitable dispersion mills include a ball mill, an attritor/attrition mill, a vibratory mill, a planetary mill, media mills, such as a sand mill and a bead mill. A media mill is preferred due to the relatively shorter milling time required to provide the desired reduction in particle size. The beads preferably are ZrO₂ beads. For instance, for the nano-particles, the ideal bead size is about 0.5 mm and, for the microparticles, the ideal bead size is about 2 mm.

The grinding media for the particle size reduction step can be selected from rigid media preferably spherical or particulate in form having an average size less than 3 mm and, more preferably, less than 1 mm (as low as 200 μm beads). Such media desirably can provide the particles of the invention with shorter processing times and impart less wear to the milling equipment. Examples of grinding media are ZrO₂ such as 95% ZrO₂ stabilized with magnesia or stabilized with yttrium, zirconium silicate, glass grinding media, polymeric beads, stainless steel, titania, alumina and the like. Preferred grinding media have a density greater than 2.5 g/cm³ and include 95% ZrO₂ stabilized with magnesia and polymeric beads.

The attrition time can vary widely and depends primarily upon the particular mechanical means and processing conditions selected. For rolling mills, processing times of up to two days or longer may be required.

The particles should be reduced in size at a temperature that does not significantly degrade the active ingredient, e.g. bedaquiline, compound. Processing temperatures of less than 30 to 40° C. are ordinarily preferred. If desired, the processing equipment may be cooled with conventional cooling equipment. The method is conveniently carried out under conditions of ambient temperature and at processing pressures, which are safe and effective for the milling process.

The pharmaceutical compositions according to the present invention contain an aqueous carrier that preferably is pharmaceutically acceptable. Said aqueous carrier comprises sterile water optionally in admixture with other pharmaceutically acceptable ingredients. The latter comprise any ingredients for use in injectable formulations. Such ingredients are optional. These ingredients may be selected from one or more of a suspending agent, a buffer, a pH adjusting agent, a preservative, an isotonizing agent, and the like ingredients. In one embodiment, said ingredients are selected from one or more of a suspending agent, a buffer, a pH adjusting agent, and optionally, a preservative and an isotonizing agent. Particular ingredients may function as two or more of these agents simultaneously, e.g. behave like a preservative and a buffer, or behave like a buffer and an isotonizing agent.

Suitable optional buffering agents and pH adjusting agents should be used in amount sufficient to render the dispersion neutral to very slightly basic (up to pH 8.5), preferably in the pH range of 7 to 7.5. Particular buffers are the salts of week acids. Buffering and pH adjusting agents that can be added may be selected from tartaric acid, maleic acid, glycine, sodium lactate/lactic acid, ascorbic acid, sodium citrates/citric acid, sodium acetate/acetic acid, sodium bicarbonate/carbonic acid, sodium succinate/succinic acid, sodium benzoate/benzoic acid, sodium phosphates, tris(hydroxymethyl)aminomethane, sodium bicarbonate/sodium carbonate, ammonium hydroxide, benzene sulfonic acid, benzoate sodium/acid, diethanolamine, glucono delta lactone, hydrochloric acid, hydrogen bromide, lysine, methanesulfonic acid, monoethanolamine, sodium hydroxide, tromethamine, gluconic, glyceric, gluratic, glutamic, ethylene diamine tetraacetic (EDTA), triethanolamine, including mixtures thereof. In an embodiment, the compositions of the invention do not contain a buffering agent. In an embodiment, especially when pH lowers, the compositions of the invention do contain a buffer, for example a citrate-phosphate buffer.

Suitable optional preservatives comprise antimicrobials and anti-oxidants which can be selected from the group consisting of benzoic acid, benzyl alcohol, butylated hydroxyanisole (BHA), butylated hydroxytoluene (BHT), chlorbutol, a gallate, a hydroxybenzoate, EDTA, phenol, chlorocresol, metacresol, benzethonium chloride, myristyl-7-piccolinium chloride, phenylmercuric acetate and thimerosal. Radical scavengers include BHA, BHT, Vitamin E and ascorbyl palmitate, and mixtures thereof. Oxygen scavengers include sodium ascorbate, sodium sulfite, L-cysteine, acetylcysteine, methionine, thioglycerol, acetone sodium bisulfite, isoacorbic acid, hydroxypropyl cyclodextrin. Chelating agents include sodium citrate, sodium EDTA and malic acid. In an embodiment of the invention, the compositions of the invention do not contain a perseverative.

An isotonizing agent or isotonifier may be present to ensure isotonicity of the pharmaceutical compositions of the present invention, and includes sugars such as glucose, dextrose, sucrose, fructose, trehalose, lactose; polyhydric sugar alcohols, preferably trihydric or higher sugar alcohols, such as glycerin, erythritol, arabitol, xylitol, sorbitol and mannitol. Alternatively, sodium chloride, sodium sulfate, or other appropriate inorganic salts may be used to render the solutions isotonic. These isotonifiers can be used alone or in combination. The suspensions conveniently comprise from 0 to 10% (w/v), in particular 0 to 6% of isotonizing agent. Of interest are nonionic isotonifiers, e.g. glucose, as electrolytes may affect colloidal stability. In an embodiment of the invention, the compositions of the invention contain an isotonizing agent or isotonifier, which, in a further embodiment is a nonionic isotonifier, such as a suitable sugar such as mannitol. The amount of the isotonizing agent is as hereinbefore described, but may also be added in a certain ratio compared to active ingredient (e.g. bedaquiline), for instance the w/w ratio of active ingredient (e.g. bedaquiline) and isotonizing agent (e.g. mannitol) may be between 1:1 and 10:1, for instance between about 2:1 and 8:1, especially between about 3:1 and 6:1 (e.g. about 4:1).

A desirable feature for a pharmaceutical composition of the invention relates to the ease of administration. The viscosity of the pharmaceutical compositions of the invention should be sufficiently low to allow administration by injection. In particular they should be designed so that they can be taken up easily in a syringe (e.g. from a vial), injected through a fine needle (e.g. a 20 G 1½, 21 G 1½, 22 G 2 or 22 G 1¼ needle) in not too long a time span. In one embodiment the viscosity of the compositions of the invention is below about 75 mPa·s, or below 60 mPa·s. Aqueous suspensions of such viscosity or lower usually meet the above-mentioned criteria.

Ideally, the aqueous suspensions according to the present invention will comprise as much active ingredient, e.g. bedaquiline, (or pharmaceutically acceptable salt thereof) as can be tolerated so as to keep the injected volume to a minimum, in particular from 3 to 70% (w/v), or from 3 to 60% (w/v), or from 3 to 40% (w/v), or from 10 to 40% (w/v), of active ingredient, e.g. bedaquiline, (or pharmaceutically acceptable salt thereof). In one embodiment the aqueous suspensions of the invention contain about 50%-70% (w/v) of active ingredient, e.g. bedaquiline, (or pharmaceutically acceptable salt thereof), or about 40%-60% (w/v) of active ingredient, e.g. bedaquiline, (or pharmaceutically acceptable salt thereof), or about 30%-50% (w/v) of active ingredient, e.g. bedaquiline, (or pharmaceutically acceptable salt thereof).

In one embodiment, the aqueous suspensions may comprise by weight, based on the total volume of the composition:
(a) from 10% to 70% (w/v), or from 20% to 60% (w/v), or from 20% to 50% (w/v), or from 20% to 40% (w/v) of active ingredient, e.g. bedaquiline, (or pharmaceutically acceptable salt thereof);
(b) from 0.5% to 20% (w/v), or from 2% to 15% or 20% (w/v), or from 5% to 15% (w/v) of a wetting agent (also referred to herein as a surface modifier);
(c) from 0% to 10% (w/v), or from 0% to 5% (w/v), or from 0% to 2% (w/v), or from 0% to 1% (w/v) of one or more buffering agents;
(d) from 0% to 20% (w/v), or from 2% to 15% or 20% (w/v), or from 5% to 15% (w/v) of a isotonizing agent
(e) from 0% to 2% (w/v) preservatives; and
(f) water for injection q.s. ad 100%.

In one embodiment, the aqueous suspensions may comprise by weight, based on the total volume of the composition:
(a) from 3% to 50% (w/v), or from 10% to 40% (w/v), or from 10% to 30% (w/v), of active ingredient, e.g. bedaquiline, (or pharmaceutically acceptable salt thereof);
(b) from 0.5% to 10% (w/v), or from 0.5% to 2% (w/v) of a wetting agent;
(c) from 0% to 10% (w/v), or from 0% to 5% (w/v), or from 0% to 2% (w/v), or from 0% to 1% (w/v) of one or more buffering agents;
(d) from 0% to 10% (w/v), or from 0% to 6% (w/v) of a isotonizing agent
(e) from 0% to 2% (w/v) preservatives; and
(f) water for injection q.s. ad 100%.

To the suspensions may optionally be added an amount of acid or base to bring the pH to a value of about pH 7. Suitable acids or bases are any of those that are physiologically acceptable, e.g. HCl, HBr, sulfuric acid, alkali metal hydroxides such as NaOH. In an embodiment, such acid or base need not be added to the compositions of the invention.

The present invention also concerns a pharmaceutical composition as described hereinbefore for use as a medicament in the treatment or prophylaxis of a disease or condition (e.g. as described herein).

In addition, the present invention concerns the use of a pharmaceutical composition as described herein for the preparation of a medicament for the prophylaxis or treatment of a disease or condition (e.g. as described herein).

The present invention further concerns a method of treating a subject bacterial or viral infection, said method comprising the administration of a therapeutically effective amount of a pharmaceutical composition as described herein.

As used herein, the word “substantially” does not exclude “completely” e.g. a composition which is “substantially free” from Y may be completely free from Y. Where necessary, the word “substantially” may be omitted from the definition of the invention. The term “about” in connection with a numerical value is meant to have its usual meaning in the context of the numerical value. Where necessary the word “about” may be replaced by the numerical value ±10%, or ±5%, or ±2%, or ±1%. All documents cited herein are incorporated by reference in their entirety.

The following examples are intended to illustrate the present invention and should not be construed as limiting the invention thereto.

### EXAMPLES

### Process Example: Preparation of Micro- and Nano-Suspensions

The active ingredient bedaquiline may be used as such or may be converted into a pharmaceutically acceptable salt thereof, such as a fumarate salt (for example the form used in the marketed product Sirturo®). Where referred to herein, bedaquiline is used in its non-salt form unless otherwise specified.

The prototype of the bedaquiline formulation is as follows:
Preparation of 200 and 100 mg/mL nano- and micro-suspensions.
Materials used:
Zirconium beads 0.5 mm (to aid process)
Sterile water for injection (Viaflo)
Bedaquiline (not milled/ground)
Surface modifier, including PEG4000 (or the like) and one or more other suitable surface modifiers (e.g. Tocopheryl PEG1000 succinate)—excipient(s)
Zirconium beads 2 mm (to aid process)
Mannitol (parenteral)—an excipient
Buffer (if needed) e.g. citrate-phosphate buffer

Glass bottles and ZrO₂ beads (either 0.5 mm or 2 mm, depending on the desired nano- or micro-suspensions), used as the milling media, were sterilized in an autoclave. The drug substance (quantity depending on the formulation to be prepared; see e.g. formulation/suspension below) was put into the glass bottle as well as a solution of surface modifier (e.g. PEG4000 and tocopheryl PEG1000 succinate) in water (quantity depending on the concentration required/desired; see e.g. formulation/suspension below) for injection. ZrO₂-beads with an average particle size of 500 μm or 2 mm (depending on whether a micro- or nano-suspension is required/desired) were added. The bottle was placed on a roller mill. The suspension was micronized/nanonized at 100 rpm for a period of time up to 72 hours. For instance, micronizing may be performed at 100 rpm for a period of 3 hours (or up to 3 hours) and nanonizing may be performed at 100 rpm for a period of up to 46 hours (e.g. about 40 hours). At the end of the milling process the concentrated micro- or nano-suspension was removed with a syringe and filled into vials. The resulting formulations (based on the nano-suspension and micro-suspension) are described in the following tables. Determination of the concentration was done by HPLC/UV. If needed, a dilution was made to a final concentration of 200 mg/ml of active ingredient bedaquiline. The resulting suspension was shielded from light. Other concentrations were also made and tested, including 300 mg/ml and 100 mg/ml nano- and micro-formulations.

Such formulations were (and will be) dosed intramuscular and subcutaneous in animals for PK study to investigate a possible long-acting effect (e.g. in treatment of leprosy). Physical stability of the suspensions will be followed up by measuring particle size after different storage conditions.

Certain embodiments of the formulation(s) have the following features:
Micro-suspension by using 2 mm Zr beads
Milling at 200 mg/mL (otherwise the concentration may be too high, e.g. with 300 mg/ml)
Longer milling, resulting in nano-suspension
A suitable surface modifier, for instance selected based on physical stability, e.g. a surface modifier or wetting agent as described herein.

### Reference Examples of Bedaquiline Micro-Suspensions

200 mg/ml Micro-Suspension Referred to Herein as Reference Example A (without Buffer) and Reference Examples B and C (with Buffer)

### Reference Example A

| | mg/ml |
|---|---|
| Bedaquiline | 200 |
| TPGS | 50 |
| Mannitol | 50 |
| Sterile water for injection | q.s. |

### Particle Size Distribution (PSD)

| Storage | Storage | | | |
|---|---|---|---|---|
| time | temperature (° C.) | D10 (μm) | D50 (μm) | D90 (μm) |
| 0 | | 0.820 | 1.99 | 4.96 |
| 1 month | 25° C. | 0.704 | 1.59 | 3.64 |

The PSD measurements after 1 month indicate that formulation remains relatively stable, and the Volume Density % is also depicted in FIG. 1 (where “Concept 7” refers to Reference Example A).

### Stability Test Using HPLC:

An HPLC test method was used to determine how stable the long acting injectable formulation of Reference Example A is. The purpose was to measure the amount of bedaquiline relative to two known degradants after certain periods of time at room temperature.

HPLC Procedure: Column—ProntoSIL 120-3-C18 SH, 100 mm length×3.0 mm i.d., 3 μm particle size, or equivalent; column temperature 35° C.; auto-sampler temperature 5° C.; Flow rate 0.5 mL/min; Detection UV; Wavelength 230 nm; Data Collection Time 50 minutes; Analysis Run Time 60 minutes; Injection volume 10 μl; Mobile Phase A is 0.03 M Hydrochloric Acid in Water; Mobile Phase B is Methanol/Acetonitrile/2-Propanol-45/45/10 (v/v/v).

| Time (at room | Degradant A | Degradant B | Bedaquiline |
|---|---|---|---|
| temperature) | (%, w/w) | (%, w/w) | (%, w/w) |
| Time zero | 0.49 | 0.05 | 102.5 |
| 3 months | 0.50 | 0.06 | 102.3 |
| 6 months | 0.50 | 0.08 | 103.2 |

The HPLC purity test shows that the formulation of Reference Example A is relatively stable for a long period (given that the relative amounts of degradants and bedaquiline remained stable).

### Reference Example B and C

| | Reference Example | Reference Example |
|---|---|---|
| | B mg/ml | C mg/ml |
| | (where applicable) | (where applicable) |
| Bedaquiline | 200 | 200 |
| TPGS | 50 | 50 |
| Buffer - citrate-phosphate | 0.01N (pH 6) | 0.05N (pH 6) |
| Mannitol | 50 | 50 |
| Sterile water for injection | q.s. | q.s. |

### Particle Size Distribution (PSD)

| Reference | | D10 | D50 | D90 |
|---|---|---|---|---|
| Example | Conditions | (μm) | (μm) | (μm) |
| B | After 3 hrs milling | 0.856 | 2.28 | 5.38 |
| C | After 3 hrs milling | 0.969 | 2.54 | 6.29 |
| B | After autoclaving | 1.13 | 2.29 | 4.72 |
| C | After autoclaving | 1.15 | 2.37 | 5.10 |
| B | After 1 month at 25° C. | 1.13 | 2.29 | 4.68 |
| C | After 1 month at 25° C. | 1.16 | 2.36 | 4.96 |
| B | After autoclaving | 1.12 | 2.27 | 4.65 |
| | and 1 month at 5° C. | | | |
| C | After autoclaving | 1.15 | 2.35 | 4.98 |
| | and 1 month at 5° C. | | | |

The PSD for these formulations under various conditions (including after autoclaving) indicate that the formulations remain relatively stable. This is shown in FIG. 2, where Concept 3 refers to Reference Example B and Concept 4 refers to Reference Example C.

### Example 1-a Micro-Suspension of the Invention

The suspensions of the Reference Examples all contain Vitamin E TPGS, which may not be tolerated parenterally, e.g. intramuscularly, particularly in the quantities specified (e.g. 50 mg/ml). The suspensions of the invention advantageously reduce the quantity of Vitamin E TPGS (as surface modifier), although it need not be completely replaced (e.g. as 5 mg/ml may be tolerated parenterally). PEG4000 (or polyethylene glycol 4000) is used, which can be supplied from Clariant GmbH. PEG4000 is a hydrophilic agent that can be used to increase the viscosity of the suspending vehicle and can act as a suspending agent.

### Example 1 Formulation

| | mg/ml |
|---|---|
| Bedaquiline | 200 |
| PEG4000 | 50 |
| TPGS | 5 |
| Buffer - citrate-phosphate | 0.01N (pH 6) |
| Mannitol | 50 |
| Sterile water for injection | q.s. |

In this case a buffer was added to avoid a drop in pH.

### Particle Size Distribution (PSD)

| Conditions | D10 (μm) | D50 (μm) | D90 (μm) |
|---|---|---|---|
| Before autoclaving | 1.27 | 3.29 | 10.4 |
| After autoclaving | 1.22 | 2.74 | 6.72 |

The PSD of the micro-suspension of Example 1 shows that the formulation remains relatively stable after autoclaving. This is shown in FIG. 3.

The approximate cloud point of the formulation of Example 1 was calculated to be about 105 to 110° C.

The autoclaving of the micro-suspension of Example 1 was conducted in a Systec autoclave (VX/VE series), where the present parameters are:
Sterilization temperature: 121° C. (above the calculated cloud point)
Sterilization time: 15 minutes
Unloading temperature: 80° C.

A typical autoclaving cycle—the steam generator builds up the required steam pressure and the steam flows into the sterilization chamber, after the sterilization temperature has been reached, it then remains constant for the duration of the sterilization period, and after the period has elapsed the cycles with the optional built-in cooling apparatus are cooled down until the unloading temperature has been reached.

Given that the autoclaving temperature is higher than the measured cloud point, it could be expected that particle aggregation would be seen, for instance due to phase-separation.

### Further Data on Micro-Suspension of Example 1

Particle Size Distribution (PSD) after Suspension is Subjected to Certain Conditions

| Conditions (all | D10 | D50 | D90 | |
|---|---|---|---|---|
| after autoclaving) | (μm) | (μm) | (μm) | Resuspendability |
| 18 days at 5° C. | 1.132 | 2.399 | 5.510 | Not tested |
| 1 month at 5° C. | 1.116 | 2.384 | 5.488 | 30 seconds |
| 1 month at 25° C. | 1.116 | 2.406 | 5.700 | 30 seconds |
| 1 month at 40° C. | 1.114 | 2.384 | 5.474 | 30 seconds |
| 1 month at 60° C. | 1.142 | 2.430 | 5.624 | 30 seconds |

The above data on PSD also show that the micro-suspension of Example 1 remains relatively stable after autoclaving and after further time (and at varying temperatures), which is also outlined in FIG. 4.

Re-suspendability: after autoclaving, particles can be seen at the bottom of the vessel, which must therefore be shaken. Advantageously, it was seen that, where tested, the formulation of Example 1 could easily be resuspended after shaking.

### Further Data on Micro-Suspension of Example 1

Particle Size Distribution (PSD) after Suspension is Subjected to Certain Further Conditions

| Conditions | D10 (μm) | D50 (μm) | D90 (μm) |
|---|---|---|---|
| After 4 hours milling, | 1.10 | 3.40 | 10.6 |
| before autoclaving | | | |
| After autoclaving, | 1.13 | 2.4 | 5.51 |
| 18 days at 5° C. | | | |
| 1 month at 25° C. | 1.12 | 2.41 | 5.70 |
| 3 months at 25° C. | 1.12 | 2.40 | 5.55 |
| 3 months at 60° C. | 1.20 | 2.52 | 5.82 |

It can be seen that even after autoclaving, there is a stable PSD, even up to 3 months at 60° C., which is outlined in FIG. 5.

The HPLC test method above was used to determine how stable the long acting injectable formulation of Example 1 is. Again, the purpose was to measure the amount of bedaquiline relative to known degradants/impurities after certain periods of time at room temperature and it gave the following results:

| Conditions | | | | | | |
|---|---|---|---|---|---|---|
| (all after | RRT0.15 | Degradant | Bedaquiline | RRT1.24 | Degradant | RRT1.64 |
| autoclaving) | (% w/w) | A (% w/w) | (% w/w) | (% w/w) | B (% w/w) | (% w/w) |
| Time zero, 5 C. | <0.05% | 0.13% | 73.0% | <0.05% | 0.14% | <0.05% |
| 14 days at 5° C. | <0.05% | 0.13% | 72.8% | <0.05% | 0.13% | <0.05% |
| 14 days at 40° C. | <0.05% | 0.13% | 72.8% | <0.05% | 0.13% | <0.05% |
| 14 days at 60° C. | <0.05% | 0.13% | 72.6% | <0.05% | 0.16% | <0.05% |
| 1 month at 5° C. | <0.05% | 0.12% | 72.0% | <0.05% | 0.14% | <0.05% |
| 1 month at 40° C. | <0.05% | 0.12% | 72.5% | <0.05% | 0.14% | <0.05% |
| 1 month at 60° C. | <0.05% | 0.12% | 72.6% | <0.05% | 0.17% | <0.05% |
| 3 months at 5° C. | <0.05% | 0.12% | 72.06% | <0.05% | 0.13% | <0.05% |
| 3 months at 40° C. | <0.05% | 0.12% | 71.76% | <0.05% | 0.13% | <0.05% |
| 3 months at 60° C. | <0.05% | 0.11% | 71.43% | <0.05% | 0.24% | <0.05% |

### Conclusions

A key conclusion is that, the suspensions of the Reference Example and of Example 1 are stable, as determined by PSD and purity determination in the PLC test method, even after autoclaving, after storage for a certain amount of time and at high temperatures.

A further key conclusion was that the suspensions of Example 1 were easily re-suspendable after autoclaving, even after storage for a certain amount of time and at high temperatures.

### Further Re-Suspendability Data

| | PEG4000 | Auto- | Storage | Resus- |
|---|---|---|---|---|
| Composition | mg/ml | claved | temp | pendability |
| 200 mg/ml bedaquiline; | 0 | Yes | 25° C. | Difficult |
| 10 mg/ml TPGS; 50 mg/ml | | | | |
| mannitol; 0.01N citrate- | | | | |
| phosphate buffer pH 6.0 | | | | |
| 200 mg/ml bedaquiline; | 50 | Yes | 25° C. | Easy |
| 10 mg/ml TPGS; 45 mg/ml | | | | |
| mannitol, 0.01N citrate- | | | | |
| phosphate buffer pH 6.0 | | | | |
| 200 mg/ml bedaquiline; | 50 | Yes | 25° C. | Easy |
| 5 mg/ml TPGS, 50 mg/ml | | | | |
| mannitol, 0.01N citrate- | | | | |
| phosphate buffer pH 6.0 | | | | |

The re-suspendability of the above compositions was objectively tested, after autoclaving in the above examples, by swirling the relevant composition. It was found that the composition without PEG4000 was difficult to re-suspend (here, it took more than 40 seconds to re-suspend, and required swirling and shaking), whereas the compositions with PEG4000 were relatively easy to re-suspend (here, they required less than 40 seconds of gentle swirling).

### Example 1A (Micro-Suspension)

| | Example 1A |
|---|---|
| | mg/ml (where applicable) |
| Bedaquiline | 200 |
| TPGS | 10 |
| PEG4000 | 50 |
| Mannitol | QS |
| Buffer - Citrate phosphate | 0.05M (pH 6) |

### Particle Size Distribution (PSD) and Resuspendability (Example 1A)

| | Storage | Time | PSD | | | |
|---|---|---|---|---|---|---|
| | temperature | point | Dv(10) | Dv(50) | Dv(90) | Resuspendability |
| Before | | T0 | 0.953 | 2.351 | 5.069 | Good (<10 sec shaking) |
| autoclavation | 40° C. | 3 M | 1.120 | 2.546 | 5.782 | Good (<10 sec shaking) |
| After | | T0 | 1.093 | 2.375 | 5.196 | Good (<10 sec shaking) |
| autoclavation | 25° C. | 6 M | 1.121 | 2.384 | 5.112 | Good (<10 sec shaking) |
| | 40° C. | 1 M | 1.159 | 2.450 | 5.640 | Good (<10 sec shaking) |
| | 40° C. | 3 M | 1.104 | 2.378 | 4.832 | Good (<10 sec shaking) |
| | 40° C. | 6 M | 1.139 | 2.403 | 4.962 | Good (<10 sec shaking) |
| | 50° C. | 1 M | 1.180 | 2.491 | 5.800 | Good (<10 sec shaking) |

### Example 1B (Micro-Suspension)

| | Example 1B |
|---|---|
| | mg/ml (where applicable) |
| Bedaquiline | 200 |
| TPGS | 20 |
| PEG4000 | 50 |
| Mannitol | QS |
| Buffer - Citrate phosphate | 0.05M (pH 6) |

### Particle Size Distribution (PSD) and Resuspendability (Example 1B)

| | Storage | Time | PSD | | | |
|---|---|---|---|---|---|---|
| | temperature | point | Dv(10) | Dv(50) | Dv(90) | Resuspendability |
| Before | | | | | | Good (<10 sec shaking) |
| autoclavation | 40° C. | 3 M | 0.849 | 2.238 | 4.915 | Good (<10 sec shaking) |
| After | | T0 | 0.895 | 2.117 | 4.716 | Good (<10 sec shaking) |
| autoclavation | 25° C. | 6 M | 0.882 | 2.074 | 4.376 | Good (<10 sec shaking) |
| | 40° C. | 1 M | 0.920 | 2.120 | 4.713 | Good (<10 sec shaking) |
| | 40° C. | 3 M | 0.867 | 2.098 | 4.784 | Good (<10 sec shaking) |
| | 40° C. | 6 M | 0.889 | 2.072 | 4.254 | Good (<10 sec shaking) |
| | 50° C. | 1 M | 0.940 | 2.144 | 4.856 | Good (<10 sec shaking) |

### Example 1C (Micro-Suspension)

| | Example 1C |
|---|---|
| | mg/ml (where applicable) |
| Bedaquiline | 200 |
| TPGS | 10 |
| PEG4000 | 25 |
| Mannitol | QS |
| Buffer - Citrate phosphate | 0.05M (pH 6) |

### Particle Size Distribution (PSD) and Resuspendability (Example 1C)

| | Storage | Time | PSD | | | |
|---|---|---|---|---|---|---|
| | temperature | point | Dv(10) | Dv(50) | Dv(90) | Resuspendability |
| Before | | T0 | 0.889 | 2.216 | 4.490 | Good (<10 sec shaking) |
| autoclavation | | | | | | |
| After | | T0 | 1.027 | 2.240 | 4.804 | Good (<10 sec shaking) |
| autoclavation | 25° C. | 6 M | 1.031 | 2.210 | 4.529 | Good (<10 sec shaking) |
| | 40° C. | 1 M | 1.075 | 2.276 | 4.993 | Good (<10 sec shaking) |
| | 40° C. | 3 M | 1.025 | 2.253 | 4.721 | Good (<10 sec shaking) |
| | 40° C. | 6 M | 1.058 | 2.247 | 4.540 | Good (<10 sec shaking) |
| | 50° C. | 1 M | 1.084 | 2.276 | 4.796 | Good (<10 sec shaking) |

### Example 1D (Micro-Suspension)

| | Example 1D |
|---|---|
| | mg/ml (where applicable) |
| Bedaquiline | 200 |
| Poloxamer 338 | 50 |
| PEG4000 | 50 |
| Mannitol | QS |
| Buffer - Citrate phosphate | 0.05M (pH 6) |

### Particle Size Distribution (PSD) and Resuspendability (Example 1D)

| | Storage | Time | PSD | | | |
|---|---|---|---|---|---|---|
| | temperature | point | Dv(10) | Dv(50) | Dv(90) | Resuspendability |
| Before | | T0 | 1.155 | 2.733 | 6.537 | Good (<10 sec shaking) |
| autoclavation | | | | | | |
| After | | T0 | 1.438 | 3.067 | 7.269 | Good (<10 sec shaking) |
| autoclavation | 25° C. | 6 M | 1.456 | 3.027 | 6.319 | Good (<10 sec shaking) |
| | 40° C. | 6 M | 1.493 | 3.137 | 7.005 | Good (<10 sec shaking) |

### Example 1E (Micro-Suspension)

| | Example 1E |
|---|---|
| | mg/ml (where applicable) |
| Bedaquiline | 200 |
| TPGS | 5 |
| Tween 20 | 5 |
| PEG4000 | 50 |
| Mannitol | QS |
| Buffer - Citrate phosphate | 0.05M (pH 6) |

### Particle Size Distribution (PSD) and Resuspendability (Example 1E)

| | Storage | Time | PSD | | | |
|---|---|---|---|---|---|---|
| | temperature | point | Dv(10) | Dv(50) | Dv(90) | Resuspendability |
| Before | | T0 | 0.867 | 2.002 | 4.406 | Good (<10 sec shaking) |
| autoclavation | | | | | | |
| After | | T0 | 0.978 | 2.035 | 4.319 | Good (<10 sec shaking) |
| autoclavation | 25° C. | 6 M | 0.985 | 2.006 | 3.961 | Good (<10 sec shaking) |
| | 40° C. | 6 M | 1.007 | 2.022 | 3.928 | Good (<10 sec shaking) |

### Reference Example 1F (Micro-Suspension)

| | Example 1F |
|---|---|
| | mg/ml (where applicable) |
| Bedaquiline | 200 |
| TPGS | 20 |
| PEG4000 | 50 |
| Sodium chloride | QS |
| Buffer - Citrate phosphate | 0.05M (pH 6) |

Particle Size Distribution (PSD) and resuspendability (Example 1F)

| | Storage | Time | PSD | | | |
|---|---|---|---|---|---|---|
| | temperature | point | Dv(10) | Dv(50) | Dv(90) | Resuspendability |
| Before | | T0 | 0.766 | 1.976 | 4.275 | Good (<10 sec shaking) |
| autoclavation | 40° C. | 3 M | 0.898 | 2.119 | 4.363 | Good (<10 sec shaking) |
| After | | T0 | 0.912 | 2.011 | 4.230 | Good (<10 sec shaking) |
| autoclavation | 25° C. | 6 M | 0.923 | 2.013 | 4.207 | Good (<10 sec shaking) |
| | 40° C. | 1 M | 0.961 | 2.039 | 4.200 | Good (<10 sec shaking) |
| | 40° C. | 3 M | 0.911 | 2.028 | 4.221 | Good (<10 sec shaking) |
| | 40° C. | 6 M | 0.938 | 2.033 | 4.201 | Good (<10 sec shaking) |
| | 50° C. | 1 M | 0.967 | 2.038 | 4.134 | Good (<10 sec shaking) |

### Example 2: Pharmacokinetic Studies

### Pharmacokinetic Studies in Mouse, Rat and Beagle Dog

A number of studies in mouse, rat and beagle dog are described in international patent application WO 2019/012100, which generally demonstrate that a sustained plasma concentration of bedaquiline and/or its active metabolite M2 were seen over certain periods of time (including 1 month, 3 months and 6 months) using e.g. the formulation of Reference Example A.

### Pharmacokinetic Profile in Rats

Formulations of concentrations 200 mg/mL were used in this study, and the micro-suspension of Reference Example A was used, i.e. using, in addition to the 200 mg/ml concentration of micro-particles (of the active bedaquiline), TPGS (4:1 bedaquiline. TPGS) and 50 mg/ml Mannitol in WFI (water for injection), without buffer. Bedaquiline is also referred to as TMC207.

These studies demonstrate that Reference Example A resulted in stable plasma levels over a prolonged period of time in male rats, when administered subcutaneously (SC) and intramuscularly (IM).

### Male Rats

The first experiment was performed on male rats, where each relevant 200 mg/ml nano-suspension and micro-suspension referred to above were administered subcutaneously (SC) and intramuscularly (IM) at a concentration of 40 mg/kg (0.2 mL/kg). An interim analysis was performed at 3 months and the results were followed-up at 6 months. Twelve rats were used in the study. Three rats were dosed intramuscularly (IM) with the 200 mg/ml micro-suspension (see Reference Example A). Three rats were dosed subcutaneously (SC) with the 200 mg/ml micro-suspension (see Reference Example A).

### Phase 1 of the Results—Up to 2200 Hours

FIG. 6 “Plasma kinetics of TMC207 in male rats when administered IM or SC with 200 mg/ml micro-formulation (see Reference Example A) at a dose of 40 mg/kg”

The following parameters were calculated for TMC207 (see Figure):

| | Microsuspension SC | Microsuspension IM |
|---|---|---|
| n | 3 | 3 |
| Cₘₐₓ (ng/ml) | 68.1 ± 17.6 | 215 ± 66.7 |
| Tₘₐₓ^{a} (h) | 24 | 18 |
| | (24.00-24.00) | (7.00-24.00) |
| Tₗₐₛₜ^{a} = around | 2184 | 2184 |
| 3 mths (h) | (2184-2184) | (2184-2184) |
| AUC_{0-2184 h} | 34700 ± 1770 | 91500 ± 13200 |
| (3 mths) (ng · h/ml) | | |

where applicable mean values are given (with min→max in parentheses)

### Phase 2 of the Results—Up to 4400 Hours

In all cases the plasma concentration of BDQ or M2 is calculated as the mean of the three rats in the relevant study.

Study in rats: for formulation of Reference Example A, i.e. the micro-suspension of 200 mg/ml concentration, and dosed SC at 40 mg/kg (StDev=standard deviation) and IM at 40 mg/kg

| | Plasma concentration of bedaquiline (BDQ) or its metabolite (M2) | | | | | | | |
|---|---|---|---|---|---|---|---|---|
| | SC at 40 mg/kg | | | | IM at 40 mg/kg | | | |
| Time (h) | BDQ | StDev | M2 | StDev | BDQ | StDev | M2 | StDev |
| 1 | 22.1 | 5.36 | 0.589 | NC | 139 | 33.2 | 5.11 | 2.24 |
| 4 | 36.9 | 7.42 | 6.62 | 1.69 | 172 | 47.2 | 18.8 | 5.98 |
| 7 | 40.7 | 6.27 | 8.59 | 1.46 | 185 | 24.2 | 28.7 | 7.57 |
| 24 | 68.1 | 17.6 | 24.0 | 1.14 | 212 | 70.6 | 77.3 | 23.5 |
| 168 | 16.5 | 4.84 | 9.03 | 1.81 | 98.0 | 19.1 | 91.5 | 33.1 |
| 336 | 18.1 | 3.30 | 9.28 | 2.10 | 69.7 | 10.2 | 54.9 | 16.8 |
| 504 | 22.8 | 3.96 | 9.68 | 2.85 | 52.2 | 4.05 | 37.9 | 16.5 |
| 672 | 14.7 | 0.964 | 7.32 | 1.34 | 42.6 | 6.85 | 29.5 | 14.8 |
| 840 | 15.1 | 1.74 | 7.40 | 2.46 | 33.6 | 6.39 | 22.3 | 10.6 |
| 1008 | 14.7 | 3.47 | 6.82 | 2.06 | 28.5 | 6.24 | 20.2 | 11.2 |
| 1176 | 13.2 | 2.99 | 5.96 | 1.76 | 24.1 | 7.04 | 16.6 | 9.37 |
| 1344 | 12.4 | 2.34 | 6.10 | 1.79 | 20.7 | 3.07 | 14.1 | 8.88 |
| 1512 | 12.0 | 0.917 | 5.81 | 1.81 | 19.7 | 5.98 | 13.4 | 7.16 |
| 1680 | 12.3 | 1.95 | 5.42 | 2.01 | 18.4 | 3.30 | 11.5 | 5.77 |
| 1848 | 10.6 | 0.83 | 5.18 | 1.51 | 14.3 | 1.35 | 11.4 | 6.39 |
| 2016 | 9.83 | 2.06 | 4.30 | 2.03 | 14.9 | 1.75 | 9.86 | 3.75 |
| 2184 | 10.2 | 2.42 | 4.55 | 1.36 | 12.6 | 0.755 | 8.87 | 3.37 |
| 2520 | 9.45 | 2.16 | 5.54 | 1.82 | 11.6 | 2.06 | 9.27 | 3.53 |
| 2856 | 8.26 | 0.737 | 4.78 | 1.61 | 10.5 | 2.65 | 7.49 | 3.02 |
| 3192 | 6.82 | 1.38 | 4.04 | 1.02 | 8.67 | 2.71 | 6.28 | 2.52 |
| 3528 | 6.83 | 2.27 | 4.02 | 1.05 | 6.92 | 2.09 | 5.68 | 2.79 |
| 3864 | 6.69 | 0.794 | 3.95 | 0.866 | 5.90 | 2.21 | 5.00 | 2.53 |
| 4200 | 6.41 | 1.72 | 3.49 | 0.987 | 4.41 | 2.04 | 3.74 | 2.03 |
| ≈CV % | 8-29 | | NC-47 | | 6-46 | | 26-63 | |
| T max (h) | 24 | | 24 | | 18 | | 120 | 83 |
| Cmax | 68.1 | 17.6 | 24.0 | 1.14 | 215 | 66.7 | 94.2 | 33.3 |
| (ng/mL) | | | | | | | | |
| T last (h) | 4200 | | 4200 | | 4200 | | 4200 | |
| AUClast | 50200 | 4240 | 24800 | 5520 | 109000 | 12300 | 75200 | 28700 |
| (ng*h/mL) | | | | | | | | |
| AUC₀₋₂₈₅₆ | 41000 | 2880 | 19300 | 4150 | 99200 | 13200 | 67600 | 26100 |
| (ng*h/mL) | | | | | | | | |
| AUCinf | NC | | NC | | 121000 | 11400 | 85500 | 28800 |
| (ng*h/mL) | | | | | | | | |

### Plasma Conentration Vs Time—Profiles for Reference Example A and Example 1

In the following figures, it is shown that the plasma concentration versus time profiles of the Reference Example A (labelled F4) and Example 1 (labelled F1) were studied in rats after SC injection of 40 mg/kg. The concentration of bedaquiline and its active metabolite M2 were measured.

Sustained plasma concentrations of the parent compound above the LLOQ (lower limit of quantification) were observed in all animals in all groups for the duration of the study. Within the first 28 days after SC administration, 2 plasma concentration peaks (Cₘₐₓ) of the parent compound were observed for both formulations F1 and F4. After 28 days, general converging of drug plasma concentrations to similar profiles and concentrations over time occurred for both formulations.

FIG. 7 shows plasma concentration versus time profiles of subcutaneous administered bedaquiline LAI micro-suspensions containing different surfactants (PEG4000 combined with TPGS, and TPGS) in rats.

Regarding plasma concentration-time profiles of the M2 metabolite, again sustained plasma concentrations of M2 above the LLOQ were observed in all animals for both F1 and F4.

FIG. 8 shows plasma concentration versus time profiles of bedaquiline (BDQ) metabolite after subcutaneous administration of BDQ LAI micro-suspensions containing different surfactants (PEG4000 combined with TPGS, and TPGS) in rats.

After intramuscular administration, again sustained plasma concentrations of the parent above the LLOQ was observed in all animals for both F1 and F4 formulations for the duration of the study.

FIG. 9 shows plasma concentration versus time profiles of intramuscular administered bedaquiline LAI micro-suspensions containing different surfactants (PEG 4000 combined with TPGS, and TPGS) in rats.

Similarly, sustained plasma concentrations were achieved for the metabolite after intramuscular administration.

FIG. 10 shows plasma concentration versus time profiles of bedaquiline (BDQ) metabolite after intramuscular administration of BDQ LAI micro-suspensions containing different surfactants (PEG4000 combined with TPGS, and TPGS) in rats.

Conclusion: both formulations of Reference Example A (F4) and Example 1 (F1) were effective, in their own way, in achieving sustained release of both drug and an active metabolite (M2) and were both therefore considered to be suitable for such purpose.

1. A method of administering a pharmaceutical composition to a patient by intramuscular or subcutaneous injection, wherein said pharmaceutical composition comprises a surface modifier that is a high molecular weight polyethylene glycol and an active pharmaceutical ingredient, or a pharmaceutically acceptable salt thereof, in the form of a suspension of micro- or nano-particles, wherein the PEG4000 assists in re-suspending said pharmaceutical composition after sterilization.

2. A process of re-suspending a pharmaceutical composition comprising an active pharmaceutical ingredient, or a pharmaceutically acceptable salt thereof, in the form of a suspension of micro- or nano-particles, wherein said pharmaceutical composition has undergone sterilization, the process comprising combining the sterilized pharmaceutical composition with a high molecular weight polyethylene glycol or the like.

3-4. (canceled)

5. A pharmaceutical composition for administration by intramuscular or subcutaneous injection, comprising a therapeutically effective amount of an active pharmaceutical ingredient, or a pharmaceutically acceptable salt thereof, in the form of a suspension of micro- or nano-particles comprising:
(a) an active pharmaceutical ingredient, or a pharmaceutically acceptable salt thereof, in micro- or nanoparticle form, and a surface modifier; and
(b) a pharmaceutically acceptable aqueous carrier,
wherein the surface modifier comprises a high molecular weight polyethylene glycol, and the pharmaceutical composition undergoes sterilization and re-suspension.

6. The pharmaceutical composition according to claim 5, wherein the surface modifier comprises at least 75% by weight PEG4000 and the remainder is one or more other suitable surface modifiers.

7. The pharmaceutical composition according to claim 6, wherein the one or more other suitable surface modifiers are poloxamers, α-tocopheryl polyethylene glycol succinates, polyoxyethylene sorbitan fatty acid esters, or salts of negatively charged phospholipids.

8. The pharmaceutical composition according to claim 7, wherein the other suitable surface modifiers is an α-tocopheryl polyethylene glycol succinate (TPGS).

9. The pharmaceutical composition according to claim 5, wherein the average effective particle size of the active pharmaceutical ingredient, or a pharmaceutically acceptable salt thereof, micro- or nano-particles is below about 50 μm.

10. The pharmaceutical composition according to claim 5, comprising by weight based on the total volume of the pharmaceutical composition:
(a) from 10% to 70% (w/v) of active pharmaceutical ingredient (or pharmaceutically acceptable salt thereof, but where the w/v is calculated on the basis of its non-salt form);
(b) from 0.5% to 20% (w/v) of a wetting agent or surface modifier;
(c) from 0% to 10% (w/v) of one or more buffering agents;
(d) from 0% to 20% (w/v) of a isotonizing agent
(e) from 0% to 2% (w/v) preservatives; and
(f) water for injection q.s. ad 100%.

11. A process for preparing the pharmaceutical composition of claim 5, comprising:
(a)
adding a micronized active pharmaceutical ingredient, or a pharmaceutically acceptable salt thereof, to a liquid medium to form a premix/predispersion, wherein the liquid medium contains a surface modifier comprising a high molecular weight polyethylene glycol (b) subjecting the premix/predispersion to mechanical means in the presence of a grinding medium to reduce the average effective particle size;
(c) sterilizing; and
(d) optionally re-suspending.

12. The process according to claim 11, wherein the re-suspending is performed by swirling for less than 40 seconds.

13-15. (canceled)

16. The method of claim 1, wherein the high molecular weight polyethylene glycol has a molecular weight of above 1000 to 8000.

17. The method of claim 16, wherein the high molecular weight polyethylene glycol is PEG4000.

18. The method of claim 1, wherein the active pharmaceutical ingredient is bedaquiline.

19. The method of claim 1, wherein the sterilizing is autoclaving.

20. The process of claim 2, wherein the high molecular weight polyethylene glycol has a molecular weight of above 1000 to 8000.

21. The process of claim 20, wherein the high molecular weight polyethylene glycol is PEG4000.

22. The process of claim 2, wherein the active pharmaceutical ingredient is bedaquiline.

23. The method of claim 2, wherein the sterilizing is autoclaving.

24. The process of claim 5, wherein the high molecular weight polyethylene glycol has a molecular weight of above 1000 to 8000.

25. The process of claim 24, wherein the high molecular weight polyethylene glycol is PEG4000.

26. The pharmaceutical composition of claim 5, wherein the active pharmaceutical ingredient is bedaquiline.

27. The pharmaceutical composition of claim 5, wherein the sterilizing is autoclaving.
